(19) United States
(12) Patent Application Publication
(10) Pub. No.: US 2015/0183816 A1
(43) Pub. Date: Jul. 2, 2015

(54) MODIFIED GALACTOOLIGOSACCHARIDES

(71) Applicant: GLYCOM A/S, Kgs., Lyngby (DK)

(72) Inventors: Elise Champion, Toulouse (FR); Dóra Molnár-Gábor, Budapest (HU); Gyula Dekany, Sinnamon Park (AU)

(73) Assignee: Glycom A/S, Kgs., Lyngby (DK)

(21) Appl. No.: 14/408,877

(22) PCT Filed: Jun. 24, 2013
(86) PCT No.: PCT/IB2013/055180

§ 371 Date: Dec. 17, 2014

(30) Foreign Application Priority Data
Jun. 22, 2012 (EP) 12173299.4

(51) Int. Cl. C07H 3/06; A23L 1/30; A23L 1/29; C12P 19/04; C12P 19/18

(52) U.S. Cl. CPC C07H 3/06; C12P 19/04; C12P 19/18; A23L 1/296; A23L 1/30; A23V 2002/00

(57) ABSTRACT

The invention relates to a method for making modified galactooligosaccharides comprising the step of enzymatic glycosylation, the modified galactooligosaccharides obtainable by the method and consumable products containing them.

### FIELD OF THE INVENTION

The present invention relates to a method for generating modified galactooligosaccharides (GOS), preferably with enhanced, bifidogenic activity.

### BACKGROUND OF THE INVENTION

It is a well-known fact that human milk is the best dietary source for new-born babies. It provides nutrients and energy necessary for babies to thrive and also non-digestible oligosaccharides (human milk oligosaccharides, prebiotics) which promote the colonization of microbiota like bifidobacteria and lactobacilli in the small intestine, thus establishing gut microflora with many health benefits, such as increased resistance to diarrhoea and infections, maturing the immune system and stimulating immune system activity.

It is also known that the gut microflora of formula-fed infants differs from that of the breast-fed infants. In general, the microbiota of breast-fed infants mainly contains bifidobacteria, while the microbiota of formula-fed infants is more diverse, with bifidobacteria often being the predominant species, but also containing other and less beneficial species in substantial amounts. This is presumably due to the lack of non-digestible human milk oligosaccharides in infant formulae, which act as prebiotics and thus contribute to the bifidogenic microbiota. A recent paper has published comparative studies on the bifidogenic index (microbiological and biochemical parameters resulting from the growth of bifidobacteria) concerning mother's milk and infant formulae without added prebiotics (Martinov et al. Afr. J. Biotechnol. 10, 2302 (2011)).

For better bifidogenic efficacy, the modern infant formulae introduced in the market contain galactooligosaccharides (GOS) (Torres et al. Compr. Rev. Food Sci. Food Safety 9, 438 (2010) and references cited therein). GOS are carbohydrate components that are not digestible by humans, but which have been shown to have a growth-promoting effect on bifidobacteria and lactobacilli, as they are able to ferment them. Moreover, GOS have been investigated as potential anti-inflammatory agents against IBD and IBS.

In other formulas, GOS are combined with live intestinal bacteria for better bifidogenicity (synbiotics), as in WO 00/33854.

Recently, β-galactosidases have been employed for making galacto- and heterooligosaccharides from lactose and monosaccharide acceptors, such as N-acetyl glucosamine, fucose, mannose (Schwab et al. Int. Diary J. 21, 748 (2011)), xylose, N-acetyl galactosamine or fucose (WO 2012/010597)).

Also recently, a method for providing a composition comprising sialic acid containing oligosaccharides has been disclosed (WO 2013/085384). The method comprises:
a) providing a source of non-digestible galactooligosaccharides (GOS) containing at least two terminally bonded β-linked galactose residues,
b) providing a sialic acid donor having (α2-3)-sialylated O-glycans,
c) contacting said GOS with said sialic acid donor in the presence of an enzyme having trans-sialidase activity in an enzyme reaction mixture, and
d) obtaining from said enzyme reaction mixture a fraction comprising at least 5 percent by weight of disialylated galactooligosaccharides (di-Sia-GOS) based on the dry matter.

As HMOs are not available in bulk and their large-scale microbial, enzymatic or chemical synthesis in a cost-efficient way has not yet been provided, there is still a need for other bifidogenic oligosaccharides having prebiotic properties similar to those of HMOs.

### BRIEF DESCRIPTION OF THE INVENTION

The first aspect of the invention relates to a method for making a modified galactooligosaccharide or mixture of modified galactooligosaccharides comprising at least one glycosyl residue, wherein a precursor galactooligosaccharide or mixture thereof represented by the formula (Gal)ₙ-A, wherein A is galactose or glucose, preferably glucose, and n is at least 2, is coupled to at least one glycosyl residue via the anomeric carbon atom of the glycosyl residue, to any of the monosaccharide units of said precursor galactooligosaccharide, wherein said glycosyl residue is not galactosyl, characterized in that at least one glycosyl donor is reacted with a precursor galactooligosaccharide represented by the formula (Gal)ₙ-A or a mixture thereof, wherein A and n are as defined above, under the catalysis of an enzyme capable of transferring said glycosyl moiety to said precursor galactooligosaccharide.

The second aspect of the invention relates to either a single modified galactooligosaccharide or a mixture comprising two or more modified GOS, the single compounds of which can be defined as a galactooligosaccharide comprising at least one glycosyl residue, said glycosyl residue, being different from galactosyl, is coupled, by its anomeric carbon atom, to any of the monosaccharide units of a galactooligosaccharide represented by the formula (Gal)ₙ-A, wherein A means galactose or glucose, preferably glucose, and n is at least 2.

The third aspect of the invention relates to a compound or a mixture of compounds obtained or obtainable by the method of the first aspect.

The fourth aspect of the invention relates to a compound or a mixture of compounds obtained or obtainable by the method of the first aspect for use in enhancing the bifidogenic effect of consumable products.

The fifth aspect of the invention relates to a consumable product, preferably a nutritional formulation, a pharmaceutical formulation or a food supplement, comprising a compound or a mixture of compounds obtained or obtainable by the method of the first aspect.

### DETAILED DESCRIPTION OF THE INVENTION

The term “galactooligosaccharide” or “GOS” preferably means an oligosaccharide, or a mixture of oligosaccharides having a linear or branched polygalactosyl chain consisting of at least 2 galactosyl units linked to a glucose or galactose residue at the reducing end, preferably linked with β1-4 linkage to glucose, thus forming a lactose unit (Galpβ1-4Glc). Preferably, galactooligosaccharides have a generic formula of (Gal)ₙ-A, wherein A is glucose or galactose, preferably glucose, and n is at least 2, and preferably from 2 to 15, more preferably from 2 to 10, even more preferably from 2 to 6. The (Gal)ₙ moiety represents a linear or branched polygalactopyranosyl residue wherein the galactopyranosyl units can be coupled to each other by β1-2 and/or β1-3 and/or β1-4 and/or β1-6 interglycosidic linkages, preferably β1-3 and/or β1-4 and/or β1-6 linkages. Where A means glucose, the galactose unit is linked to it preferably by β1-4 interglycosidic linkage, thus forming a lactose unit at the reducing end. The most important galactooligosaccharides are: Galpβ1-6Galpβ1-4Glc, Galpβ1-3Galpβ1-4Glc, Galpβ1-4Galpβ1-4Glc, Galpβ1-4Galpβ1-6Glc, Galpβ1-4Galpβ1-3Glc, Galpβ1-4Galpβ1-2Glc, Galpβ1-6Galpβ1-6Gal, Galpβ1-4Galpβ1-4Galpβ1-4Glc, Galpβ1-6Galpβ1-6Galpβ1-4Glc, Galpβ1-3Galpβ1-6Galpβ1-4Glc, Galpβ1-6Galpβ1-3Galpβ1-4Glc, Galpβ1-3Galpβ1-3Galpβ1-4Glc, Galpβ1-6Galpβ1-6Galpβ1-6Galpβ1-4Glc, Galpβ1-3Galpβ1-3Galpβ1-3Galpβ1-4Glc, Galpβ1-3Galpβ1-3Galpβ1-3Galpβ1-3Galpβ1-4Glc, Galβ1-3Galpβ1-3Galpβ1-3Galpβ1-3Galpβ1-3Galpβ1-4Glc.

GOS can be produced by known chemical methods, but the preferred method to synthesize them is the enzymatic approach. Two types of enzyme, namely galactosyl transferases (EC 2.4) and galactosyl hydrolases (galactosidases, EC 3.2.1), preferably β-galactosidases, are incubated in the presence of lactose. In a thermodynamically favoured reaction the galactosidase hydrolyses the lactose into glucose and galactose, and under kinetic control, i.e. when the acceptor (i.e. lactose) concentration is high enough, the enzyme transfers the galactosyl unit to the acceptor. The product thus formed can be an acceptor for further galactosyl transfer. During this enzymatic reaction, the amount and nature of the formed oligosaccharide mixture depends on several factors such as enzyme source, concentration, nature of the substrate, pH, temperature and time.

Galactosidases from Kluyveromyces, Aspergillus, Bacillus, Cryptococcus, Streptococcus and bifidobacteria are generally used for making GOS from lactose. As the solubility of lactose is relatively low at room temperature, a higher temperature can be desirable for getting higher initial lactose concentration. The use of thermostable galactosidases such as those from S. solfataricus, T. maritima, P. furiosus, T caldophylus and Thermus sp. are favourable at elevated temperature. Moreover, genetically engineered enzymes having high transgalactosidase activity and suppressed or diminished hydrolase activity can be also employed.

The polymerization degree and the structure of the GOS compounds largely depend on the specificity of the enzyme used and the reaction conditions. Generally β1-3, β1-4 and β1-6 interglycosidic linkages and mixtures thereof are the most characteristic. Commercially available GOS mixtures employed in food industry are Oligomate 55® and TOS-100® (Yakult Honsha, Japan), CUP-Oligo® (Nissin Sugar, Japan), Vivinal GOS® (Friesland Foods Domo, The Netherlands), Bimuno® (Clasado, UK), Purimune® (Corn Products Intl., USA) and Promovita GOS® (Fayrefield Food and First Milk, both UK).

The term “fucosyl” preferably means a L-fucopyranosyl group attached to the core oligosaccharide with α-interglycosidic linkage:

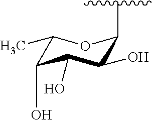

“N-acetyl-glucosaminyl” preferably means an N-acetyl-2-amino-2-deoxy-D-glucopyranosyl (GlcNAc) group linked with β-linkage:

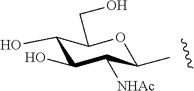

“N-acetyl-lactosaminyl” preferably means the glycosyl residue of N-acetyl-lactosamine (LacNAc, Galpβ1-4GlcNAcp) linked with β-linkage:

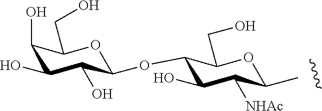

Furthermore, the term “lacto-N-biosyl” preferably means the glycosyl residue of lacto-N-biose (LNB, Galpβ1-3GlcNAcp) linked with β-linkage:

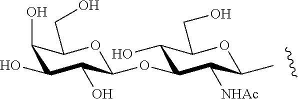

The term “sialyl” preferably means the glycosyl residue of sialic acid (N-acetyl-neuraminic acid, Neu5Ac) linked with α-linkage:

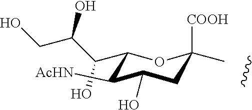

The term “optionally substituted phenoxy” preferably means a phenoxy group optionally substituted with 1 or 2 groups selected from nitro, halogen, alkyl, hydroxyalkyl, amino, formyl, carboxyl and alkoxycarbonyl, or two substituents in ortho position can form a methylenedioxy-group. Preferred substituents are nitro (preferably in 2- and/or 4-position), halogen (preferably fluoro and chloro). Especially preferred substituted phenoxy groups are selected from 4-nitrophenoxy, 2,4-dinitrophenoxy, 2-chloro-4-nitrophenoxy, 2-fluoro-4-nitrophenoxy, 3-fluoro-4-nitrophenoxy, 2-hydroxymethyl-4-nitrophenoxy, 3-hydroxymethyl-4-nitrophenoxy, 2-formyl-4-nitrophenoxy, 2-carboxy-4-nitrophenoxy, 2-methoxycarbonyl-4-nitrophenoxy, 5-fluoro-2-nitrophenoxy, 4-methoxycarbonyl-2-nitrophenoxy, 4-carboxy-2-nitrophenoxy, 2-aminophenoxy and 3,4-methylenedioxy-phenoxy.

The term “optionally substituted pyridinyloxy” preferably means a pyridinyloxy, more preferably 2- or 4-pyridyloxy group, optionally substituted with 1 or 2 groups selected from nitro, halogen, alkyl and alkoxy. Especially preferred pyridinyloxy groups are selected from 4-pyridinyloxy, 2-pyridinyloxy, 3-nitro-2-pyridinyloxy and 3-methoxy-2-pyridinyloxy.

The term “donor” is to be understood as a compound that provides or transfers a specific moiety in a chemical reaction, e.g. a nucleophilic or electrophilic substitution reaction, to a further compound, preferably an acceptor. Particularly preferably, a “donor” is understood as a compound that provides or transfers a glycosyl residue to a further compound, preferably an acceptor, wherein the donor is not restricted to naturally occurring donors.

Likewise, the term “acceptor” is to be understood as a compound that receives a specific moiety, preferably a glycosyl moiety, in a chemical reaction, e.g. nucleophilic or electrophilic substitution reaction, from a further compound, preferably a donor as defined above.

According to a first aspect, the present invention provides a method for making a modified galactooligosaccharide or mixture of modified galactooligosaccharides comprising at least one glycosyl residue, wherein a precursor galactooligosaccharide or mixture thereof represented by the formula (Gal)ₙ-A, wherein A is galactose or glucose, preferably glucose, and n is at least 2, is coupled to at least one glycosyl residue via the anomeric carbon atom of the glycosyl residue, to any of the monosaccharide units of said precursor galactooligosaccharide, wherein said glycosyl residue is not galactosyl, characterized in that at least one glycosyl donor is reacted with a precursor galactooligosaccharide represented by the formula (Gal)ₙ-A or a mixture thereof, wherein A and n are as defined above, under the catalysis of an enzyme capable of transferring said glycosyl moiety to said precursor galactooligosaccharide.

According to a preferred embodiment of the first aspect, the method comprises the steps of:
a) providing at least one glycosyl donor,
b) providing a precursor galactooligosaccharide,
c) providing at least one enzyme comprising a trans-glycosidase or a glycosynthase activity;
d) preparing a mixture of the components provided in steps a), b) and c);
e) incubating the mixture prepared according to step d);
f) optionally: repeating steps a), c), d) and e) with the mixture obtained according to step e).

According to step a) at least one glycosyl donor is provided, and the glycosyl donor is not a galactosyl donor. Compounds for use as glycosyl donors in step a) are preferably selected from the group consisting of: a sialyl donor, a fucosyl donor and an optionally galactosylated N-acetyl-glucosaminyl donor. More preferably, said sialyl donor, fucosyl donor and/or optionally galactosylated N-acetyl-glucosaminyl donor has a leaving group selected from the group consisting of: fluoro, azido and —OR group, wherein R can be a mono-, di- or oligosaccharide, glycolipid, glycoprotein or glycopeptide, cyclic or acyclic aliphatic group, or aryl residue; or wherein the optionally galactosylated N-acetyl-glucosaminyl donor is an oxazoline. Even more preferably said sialyl donor is characterized by formula 1, said fucosyl donor is characterized by formula 2 and said optionally galactosylated N-acetyl-glucosaminyl donor is characterized by formulae 3 or 4

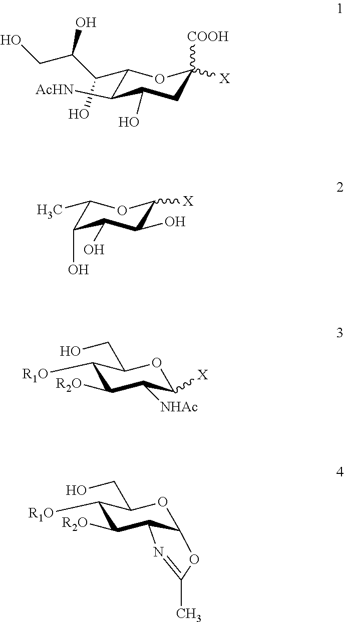

wherein X, independently, is selected from the group consisting of azide, fluoro, optionally substituted phenoxy, optionally substituted pyridinyloxy, lactose moiety, group A, group B, group C and group D

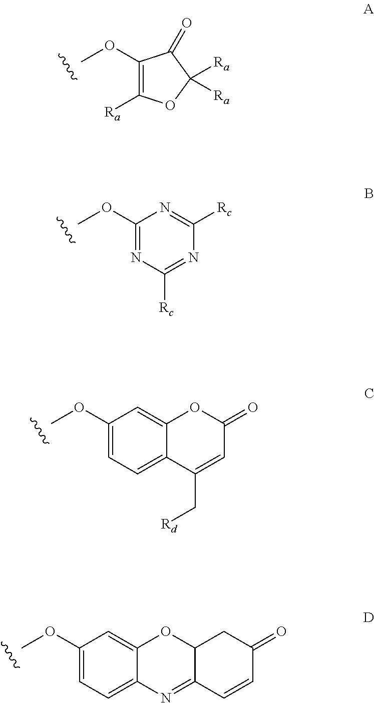

wherein Rₐ is independently H or alkyl, or two vicinal Rₐ groups represent a ═C(R_{b})₂ group, wherein R_{b} is independently H or alkyl, R_{c} is independently selected from the group consisting of alkoxy, amino, alkylamino and dialkylamino, R_{d} is selected from the group consisting of H, alkyl and —C(═O)Rₑ, wherein Rₑ is OH, alkoxy, amino, alkylamino, dialkylamino, hydrazino, alkylhydrazino, dialkylhydrazino or trialkylhydrazino;
and R₁ and R₂, independently, is H or β-D-galactopyranosyl group with the proviso that at least one of the R₁ and R₂ groups is H.

Compounds for use as glycosyl donors in this step a) are preferably selected from the group consisting of: a sialyl donor, a fucosyl donor and an optionally galactosylated N-acetyl-glucosaminyl donor. More preferably, said sialyl donor, fucosyl donor and/or optionally galactosylated N-acetyl-glucosaminyl donor has a leaving group selected from the group consisting of: fluoro, azido and —OR group, wherein R can be a mono-, di- or oligosaccharide, glycolipid, glycoprotein or glycopeptide, cyclic or acyclic aliphatic group, or aryl residue; or wherein the optionally galactosylated N-acetyl-glucosaminyl donor is an oxazoline. Even more preferably said sialyl donor is characterized by formula 1, said fucosyl donor is characterized by formula 2 and said optionally galactosylated N-acetyl-glucosaminyl donor is characterized by formulae 3 or 4 wherein X is as defined above.

The donors are preferably selected with reference to the enzymes used during the method of the present invention. Preferably, the donors are selected depending on the enzyme's transglycosidase activity from compounds according to formulae 1 to 3, wherein X is optionally substituted 4-nitrophenoxy, lactose moiety, group A, group B, group C, or from compounds of formula 4. Likewise preferably, the donors are selected depending on the enzyme's glycosynthase activity from compounds according to formulae 1 to 3, wherein X is azide or fluoro. Both selections can be carried out independently of each other or together.

Furthermore, compounds for use as donors in step a) can preferably be selected from compounds according to formulae 1 to 3, wherein X is lactose moiety, 4-nitrophenoxy, 2,4-dinitrophenoxy, 2-chloro-4-nitrophenoxy, 2,5-dimethyl-3-oxo-(2H)-furan-4-yloxy, 2-ethyl-5-methyl-3-oxo-(2H)-furan-4-yloxy, 5-ethyl-2-methyl-3-oxo-(2H)-furan-4-yloxy, 4,6-dimethoxy-1,3,5-triazin-2-yloxy, 4,6-diethoxy-1,3,5-triazin-2-yloxy, 4-methylumbelliferyloxy, or from compounds of formula 4 represented by formulae 5, 6 or 7.

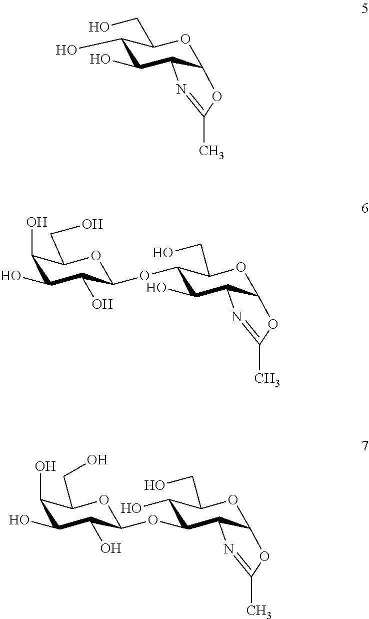

It is in some circumstances preferred that in step a) the at least one glycosyl donor is not a sialyl donor selected from synthetic sialic acid glycosides (such as 2′-(4-methylumbelliferyl)-α-N-acetylneuraminic acid), or not a sialyl donor selected from α2-3-sialylated glycoprotein, glycopeptide and glycolipid, such as glycomacropeptide from κ-casein (GMP), pig small intestinal glycoprotein (PSMG) or fragments thereof, or obtained from mucin, if the enzyme provided in step c) is a trans-sialidase having α-2,3 activity, such as a transsialidase derived from a Trypanosoma species, in particular T. cruzi. Compound for use as sialyl donor in this step a) is preferably α2-3-sialylated monosaccharide, oligosaccharide or polysaccharide, particularly 3′-SL.

In step c) at least one enzyme comprising a transglycosidase activity and/or a glycosynthase activity is provided.

Enzymes suitable in step c) typically comprise at least one enzyme comprising a transglycosidase activity and/or a glycosynthase activity, preferably selected from enzymes having, e.g. a fucosidase, trans-fucosidase or fucosynthase activity, a sialidase (neuraminidase) or trans-sialidase (transneuraminidase) activity, a N-acetylglucosaminidase or trans-N-acetylglucosaminidase activity, a lacto-N-biosidase or trans-lacto-N-biosidase activity and/or a N-acetyllactosaminidase or trans-N-acetyllactosaminidase activity, or any further enzyme having such an activity. Even more preferably, enzymes suitable in step c) can be selected from the group comprising wild type or mutated glycosidases or transglycosidases, preferably wild type or mutated glycosidases or transglycosidases having a fucosidase, trans-fucosidase or fucosynthase activity, a sialidase (neuraminidase) or trans-sialidase (transneuraminidase) activity, a N-acetylglucosaminidase or trans-N-acetylglucosaminidase activity, a lacto-N-biosidase or trans-lacto-N-biosidase activity and/or a N-acetyllactosaminidase or trans-N-acetyllactosaminidase activity, or preferably having α-trans-fucosidase, α-trans-sialidase, β-trans-N-acetylglucosaminidase, β-trans-lacto-N-biosidase and/or β-trans-N-acetyllactosaminidase activity.

The source of the enzymes suitable in step c) furthermore can be selected from any genus known to a skilled person to express or secrete at least one enzyme as defined above, e.g. an enzyme having a transglycosidase activity and/or a glycosynthase activity, preferably an enzyme having a fucosidase, trans-fucosidase or fucosynthase activity, a sialidase (neuraminidase) or trans-sialidase (transneuraminidase) activity, a N-acetylglucosaminidase or trans-N-acetylglucosaminidase activity, a lacto-N-biosidase or trans-lacto-N-biosidase activity and/or a N-acetyllactoaminidase or trans-N-acetyllactoaminidase activity, or preferably having α-trans-fucosidase, α-trans-sialidase, β-trans-N-acetylglucosaminidase, β-trans-lacto-N-biosidase and/or β-trans-N-acetyllactosaminidase activity, or any further enzyme having such an activity. Even more preferably, the source of the enzymes suitable in step c) can be selected from non-pathogenic bacteria selected from Bacillus, Bifidobacterium, Lactobacillus, Leuconostoc, Lactococcus, Streptococcus, Streptomyces, Sulfolobus, Thermotoga, or Trypanosoma.

According to another embodiment with regard to the suitable enzymes in step c), where the donor provided in step a) is a sialyl donor selected from α2-3-sialylated monosaccharide, oligosaccharide, polysaccharide, glycoprotein, glycopeptide and glyco lipid, such as glycomacropeptide from κ-casein (GMP), pig small intestinal glycoprotein (PSMG) or fragments thereof, or obtained from mucin, preferably the enzymes can be selected from those having, e.g. a fucosidase, trans-fucosidase or fucosynthase activity, a sialidase (neuraminidase) or trans-sialidase (transneuraminidase) activity (provided that sialidase or trans-sialidase activity is not a α2-3-sialidase or α2-3-trans-sialidase activity, such as a transsialidase derived from a Trypanosoma species, in particular T. cruzi), a N-acetylglucosaminidase or trans-N-acetylglucosaminidase activity, a lacto-N-biosidase or trans-lacto-N-biosidase activity and/or a N-acetyllactosaminidase or trans-N-acetyllactosaminidase activity, or any further enzyme having such an activity. Even more preferably, enzymes suitable in this step c) can be selected from the group comprising wild type or mutated glycosidases or transglycosidases, preferably wild type or mutated glycosidases or transglycosidases having a fucosidase, trans-fucosidase or fucosynthase activity, a sialidase (neuraminidase) or trans-sialidase (transneuraminidase) activity, a N-acetylglucosaminidase or trans-N-acetylglucosaminidase activity, a lacto-N-biosidase or trans-lacto-N-biosidase activity and/or a N-acetyllactosaminidase or trans-N-acetyllactosaminidase activity, or preferably having α-trans-fucosidase, α-trans-sialidase, β-trans-N-acetylglucosaminidase, β-trans-lacto-N-biosidase and/or β-trans-N-acetyllactosaminidase activity. The source of the enzymes suitable in this step c) furthermore can be selected from any genus known to a skilled person to express or secrete at least one enzyme as defined above, e.g. an enzyme having a transglycosidase activity and/or a glycosynthase activity, preferably an enzyme having a fucosidase, trans-fucosidase or fucosynthase activity, a sialidase (neuraminidase) or trans-sialidase (transneuraminidase) activity (except for a sialidase (neuraminidase) or trans-sialidase (transneuraminidase) activity with α2-3 selectivity), a N-acetylglucosaminidase or trans-N-acetylglucosaminidase activity, a lacto-N-biosidase or trans-lacto-N-biosidase activity and/or a N-acetyllactosaminidase or trans-N-acetyllactosaminidase activity, or preferably having α-trans-fucosidase, α-trans-sialidase, β-trans-N-acetylglucosaminidase, β-trans-lacto-N-biosidase and/or β-trans-N-acetyllactosaminidase activity, or any further enzyme having such an activity. Even more preferably, the source of the enzymes suitable in step c) can be selected from non-pathogenic bacteria selected from Bacillus, Bifidobacterium, Lactobacillus, Leuconostoc, Lactococcus, Streptococcus, Streptomyces, Sulfolobus, Thermotoga, or Trypanosoma.

Even more preferably, the source of the enzymes suitable in step c) is selected from the group comprising the non-pathogenic bacteria Bacillus circulans, lactic acid bacteria, such as Bifidobacterium bifidum JCM 1254, Bifidobacterium bifidum NCIMB 41171, Bifidobacterium bifidum NCIMB 41171, Bifidobacterium bifidum JCM1254, Bifidobacterium bifidum JCM1254, Bifidobacterium bifidum PRL2010, Bifidobacterium bifidum PRL2010, Bifidobacterium bifidum S17, Bifidobacterium bifidum S17, Bifidobacterium dentium Bd1, Bifidobacterium longum subsp. infantis ATCC 15697, Bifidobacterium longum subsp longum JDM 301, Bifidobacterium longum subsp. infantis JCM 1222, Lactobacillus casei BL23, Streptomyces sp., Sulfolobus solfataricus P2, Thermotoga maritima MSB8, and Trypanosoma cruzi.

Particularly preferred microorganisms in the above context, particularly for targeted glycosidases/transglycosidases/glycosynthases, comprise lactic acid bacteria. Lactic acid bacteria, and more particularly non-pathogenic bacteria from the genus Bifidobacterium contain a series of glycosidases including α-2,6 sialidases (GH33), α-1,2/3/4 fucosidases (GH29 and GH95), lacto-N-biosidases (GH20) and β-N-acetylhexosaminidases (GH18, GH20, GH56, GH84, GH85 and GH123) that are able to recognize GOS and/or human milk oligosaccharides. Depending on the bifidobacteria strains, these glycosidases are intra- or extracellular enzymes.

A further aspect regarding the use of glycosidases and/or glycosynthases from lactic acid bacteria concerns the industrial importance of such bacteria since they have the GRAS (generally recognized as safe) status. According to another more preferred aspect the glycosidase and/or glycosynthases displaying a trans-fucosidase, trans-sialidase, trans-N-acetylglucosaminidase, trans-lacto-N-biosidase and/or trans-N-acetyllactosaminidase activity, preferably a α-trans-fucosidase, α-trans-sialidase, β-trans-N-acetylglucosaminidase, β-trans-lacto-N-biosidase and/or β-trans-N-acetyllactosaminidase activity, is a wild type or an engineered glycosidase, and most preferably a wild type glycosidase is obtained from the group consisting of lactic acid bacteria, wherein the glycosidase is converted to a transglycosidase by rational engineering or/and directed evolution. A glycosidase and/or glycosynthase obtained from the group consisting of lactic acid bacteria is most preferably a glycosidase from Bifidobacterium, Lactobacillus, Lactococcus, Streptococcus or Leuconostoc. A glycosidase selected from the genus Bifidobacterium is most preferably a glycosidase from Bifidobacterium longum subsp. Infantis, Bifidobacterium longum subsp. Longum, Bifidobacterium breve, Bifidobacterium bifidum and Bifidobacterium catenulatum.

Furthermore, engineered fucosidases from thermophilic organisms such as Sulfolobus solfataricus and Thermotoga maritima have recently been developed, which can be used in the method of the present invention. These thermostable glycosidases have considerable potential for industrial applications since they can be used in biotechnological processes at elevated temperatures, so facilitating the process, preventing risk of contamination, and increasing the solubility of the compounds used in the reaction.

According to another more preferred embodiment, the glycosidase and/or glycosynthase enzyme displaying a trans-fucosidase, trans-sialidase, trans-N-acetylglucosaminidase, trans-lacto-N-biosidase and/or trans-N-acetyllactosaminidase activity, preferably an α-trans-fucosidase, α-trans-sialidase, β-trans-N-acetylglucosaminidase, β-trans-lacto-N-biosidase and/or β-trans-N-acetyllactosaminidase activity, is a wild type or an engineered glycosidase. Most preferably, the wild type glycosidase is obtained from the group consisting of thermophilic organisms, which glycosidase is converted to a transglycosidase by rational engineering or/and directed evolution. An α-L-fucosidase obtained from thermophilic organisms is most preferably an α-L-fucosidase from Thermotoga maritima and Sulfolobus solfataricus.

Preferably, the at least one enzyme comprising a transglycosidase activity and/or a glycosynthase activity can be selected from an enzyme exhibiting a fucosidase, trans-fucosidase or fucosynthase activity, preferably as described below. In this context, enzymes having a fucosidase, trans-fucosidase or fucosynthase activity, more preferably an α-trans-fucosidase activity, are preferably selected from fucosidases in general, and more preferably from α-L-fucosidases, e.g. α-L-fucosidases as classified according to EC 3.2.1.38 and 3.2.1.51. α-L-Fucosidases are widely spread in living organisms such as mammals, plants, fungi and bacteria. These enzymes belong to the families 29 and 95 of the glycoside hydrolases (GH29 and GH95) as defined by the CAZY nomenclature (http://www.cazy.org).

Fucosidases from GH29 are retaining enzymes (3D structure: (β/α)₈) whereas fucosidases from GH95 are inverting enzymes (3D structure: (α/α)₆). The substrate specificity of the GH29 family is broad whereas that of the GH95 family is strict to α1,2-linked fucosyl residues. The GH29 family seems to be divided into two subfamilies. One subfamily typically has strict specificity towards α1,3- and α1,4-fucosidic linkages. The members of a further subfamily have broader specificity, covering all α-fucosyl linkages. α-L-fucosidases generally hydrolyse the terminal fucosyl residue from glycans. These enzymes are also capable of acting as catalysts for fucosylation reactions due to their transfucosylation activity and thus can be used in the context of the method of the present invention, preferably under kinetically controlled conditions.

Fucosidases, which can be employed in the context of the present invention, can also comprise engineered fucosidases. Such engineered fucosidases preferably comprise engineered α-L-fucosidases, preferably engineered fucosidases derived from fucosidases as described above, e.g. an engineered α-1,2-L-fucosynthase from Bifidobacterium bifidum, α-L-fucosynthases from Sulfolobus solfataricus and Thermotoga maritime, etc. Such engineered fucosidases show an acceptor dependent regioselectivity and are devoid of product hydrolysis activity. Furthermore, engineered fucosidases preferably comprise α-L-fucosidase from Thermotoga maritima, which has also been recently converted into an efficient α-L-trans-fucosidase by directed evolution (see Osanjo et al. Biochemistry 46, 1022 (2007)).

Even more preferably, the at least one enzyme having a fucosidase and/or trans-fucosidase and/or fucosynthase activity can be selected from α-L-fucosidases derived from Thermotoga maritima MSB8, Sulfolobus solfataricus P2, Bifidobacterium bifidum JCM 1254, Bifidobacterium bifidum JCM 1254, Bifidobacterium longum subsp. infantis ATCC 15697, Bifidobacterium longum subsp. infantis ATCC 15697, Bifidobacterium longum subsp. Infantis JCM 1222, Bifidobacterium bifidum PRL2010, Bifidobacterium bifidum S17, Bifidobacterium longum subsp longum JDM 301, Bifidobacterium dentium Bdl, or Lactobacillus casei BL23, etc.

Even more preferably, the at least one enzyme having a fucosidase and/or trans-fucosidase and/or fucosynthase activity can be selected from following α-L-fucosidases as defined according to the following deposit numbers gi|4980806 (Thermotoga maritima MSB8), gi|13816464 (Sulfolobus solfataricus P2), gi|34451973 (Bifidobacterium bifidum JCM 1254), gi|242345155 (Bifidobacterium bifidum, JCM 1254), gi|213524647 (Bifidobacterium longum subsp. infantis, ATCC 15697), gi|213522629 (Bifidobacterium longum subsp. infantis ATCC 15697), gi|213522799 (Bifidobacterium longum subsp. infantis ATCC 15697), gi|213524646 (Bifidobacterium longum subsp. infantis ATCC 15697), gi|320457227 (Bifidobacterium longum subsp. infantis JCM 1222), gi|320457408 (Bifidobacterium longum subsp. infantis JCM 1222), gi|320459369 (Bifidobacterium longum subsp. infantis JCM 1222), gi|320459368 (Bifidobacterium longum subsp. infantis JCM 1222), gi|310867039 (Bifidobacterium bifidum PRL2010), gi|310865953 (Bifidobacterium bifidum PRL2010), gi|309250672 (Bifidobacterium bifidum S17), gi|309251774 (Bifidobacterium bifidum S17), gi|296182927 (Bifidobacterium longum subsp longum JDM 301), gi|296182928 (Bifidobacterium longum subsp longum JDM 301), gi|283103603 (Bifidobacterium dentium Bdl), gi|190713109 (Lactobacillus casei BL23), gi|190713871 (Lactobacillus casei BL23), gi|190713978 (Lactobacillus casei BL23), etc., or a sequence exhibiting a sequence identity with one of the above mentioned enzyme sequences having a fucosidase and/or trans-fucosidase activity of at least 70%, more preferably at least 80%, more preferably at least 85%, even more preferably at least 90% and most preferably at least 95% or even 97%, 98% or 99% as compared to the entire wild type sequence on amino acid level.

Wild type or engineered fucosidases as defined above, displaying transfucosidase activity and showing a α1-2, α1-3 and/or α1-4 regioselectivity, can be used in the present invention. Such wild type or engineered fucosidases preferably display transfucosidase activity and catalyse the transfer of the fucosyl residue to:
a galactosyl moiety of the polygalactosyl chain, preferably to a terminal galactosyl moiety of that chain, with 1-2 or 1-3 interglycosidic linkage and/or
the galactose or glucose of moiety A with 1-2 or 1-3 interglycosidic linkage.

Additionally, wild type or engineered fucosidases as defined above, displaying transfucosidase activity and showing a α1-2, α1-3 and/or α1-4 regioselectivity, can catalyse the transfer of the fucosyl residue to:
the galactose of the lacto-N-biosyl group with 1-2 interglycosidic linkage and/or
the N-acetyl-glucosamine of the lacto-N-biosyl group with 1-4 interglycosidic linkage and/or
the N-acetyl-glucosamine of the N-acetyl-lactosaminyl group with 1-3 interglycosidic linkage and/or
a N-acetylglucosaminyl moiety, preferably to a terminal N-acetylglucosaminyl moiety with 1-3 or 1-4 interglycosidic linkage,
   provided that a lacto-N-biosyl, N-acetyl-lactosaminyl or N-acetylglucosaminyl group is already present in a modified GOS as a result of an antecedent enzymatic transfer of these glycosyl groups within the frame of the method of the present invention.

Also preferably, the regioselectivity the α-L-fucosidases with fucosidase/trans-fucosidase/fucosynthase activity used in the method of this invention matches the fucosyl donor of formula 2 provided in step a) when X means a lactose moiety. In this regard, when 2′-FL is added in step a), then a α1-2-L-fucosidase with fucosidase/trans-fucosidase/fucosynthase activity is preferably provided in step c), and when 3-FL is added in step a), then a α1-3-L-fucosidase with fucosidase/trans-fucosidase/fucosynthase activity is preferably provided in step c).

Particularly preferred α-L-fucosidases or the wild types of engineered fucosidases with fucosidase/trans-fucosidase/fucosynthase activity are listed in the following Table 1:

TABLE 1
Preferred α-L-fucosidases

| GI number | |
|---|---|
| in GenBank | |
| Database | Organisms |
| gi\|4980806 | Thermotoga maritima MSB8 |
| gi\|13816464 | Sulfolobus solfataricus P2 |
| gi\|34451973 | Bifidobacterium bifidum JCM 1254 |
| gi\|242345155 | Bifidobacterium bifidum JCM 1254 |
| gi\|213524647 | Bifidobacterium longum subsp. infantis ATCC 15697 |
| gi\|213522629 | Bifidobacterium longum subsp. infantis ATCC 15697 |
| gi\|213522799 | Bifidobacterium longum subsp. infantis ATCC 15697 |
| gi\|213524646 | Bifidobacterium longum subsp. Infantis ATCC 15697 |
| gi\|320457227 | Bifidobacterium longum subsp. infantis JCM 1222 |
| gi\|320457408 | Bifidobacterium longum subsp. infantis JCM 1222 |
| gi\|320459369 | Bifidobacterium longum subsp. infantis JCM 1222 |
| gi\|320459368 | Bifidobacterium longum subsp. infantis JCM 1222 |
| gi\|310867039 | Bifidobacterium bifidum PRL2010 |
| gi\|310865953 | Bifidobacterium bifidum PRL2010 |
| gi\|309250672 | Bifidobacterium bifidum S17 |
| gi\|309251774 | Bifidobacterium bifidum S17 |
| gi\|296182927 | Bifidobacterium longum subsp longum JDM 301 |
| gi\|296182928 | Bifidobacterium longum subsp longum JDM 301 |
| gi\|283103603 | Bifidobacterium dentium Bd1 |
| gi\|190713109 | Lactobacillus casei BL23 |
| gi\|190713871 | Lactobacillus casei BL23 |
| gi\|190713978 | Lactobacillus casei BL23 |

Likewise preferably, the at least one enzyme comprising a transglycosidase activity and/or a glycosynthase activity can be selected from an enzyme exhibiting a sialidase or trans-sialidase activity, preferably as described in the following. In this context, enzymes having a sialidase or trans-sialidase activity are preferably selected from a sialidase or trans-sialidase as described in the following, e.g. sialidases (EC 3.2.1.18) and trans-sialidases (EC 2.4.1.-) as classified according to the the GH33 family. They are retaining enzymes. Sialidases and trans-sialidases are widely distributed in nature. They are found particularly in diverse virus families and bacteria, and also in protozoa, some invertebrates and mammals. These enzymes differ in their biochemical properties, e.g., kinetics, binding affinity or substrate preference. Nevertheless, they possess conserved domains and structural similarities. Trans-sialidases differ from sialidases since they can transfer sialic acids, preferably α-2,3-bonded sialic acids, from a donor molecule to an acceptor derivative, which is preferably a terminal galactose moiety with a β-interglycosidic linkage. As a result of this transfer, an α-glycosidic bond is formed between the sialic acid and the acceptor. However, if there is no suitable acceptor, the trans-sialidase hydrolyses the sialic acid.

The first trans-sialidase enzyme described was found in Trypanosoma cruzi, a protozoa which causes Chagas disease. This trans-sialidase (TcTS) has been extensively studied. Since that time trans-sialidases have been detected in several other trypanosome types such as Trypanosoma brucei gambiense, Trypanosoma brucei rhodesiense, Trypanosoma brucei brucei and Trypanosoma congolense. Moreover, the existence of trans-sialidases has been shown in Endotrypanum types, in Corynebacterium diphtheriae and even in human plasma.

Sialidases can be classified into two different subgroups, endo- and exo-sialidases. The endo-sialidases hydrolyse sialic acid linkages internal to macromolecules, while the exo-sialidases attack terminal sialic acid linkages, and desialylate glycoproteins, glycopeptides, gangliosides, oligosaccharides and polysaccharides. Recently, sialidases from Bifidobacterium bifidum and Bifidobacterium longum subsp. infantis have been identified, cloned and characterized. These sialidases can cleave and so recognize both α-2,3- and α-2,6-linked sialosides. Sialidases from Bifidobacterium longum subsp. infantis have a consistent preference for α-2,6-linkage whereas sialidases from Bifidobacterium bifidum have a consistent preference for α-2,3-linkage. These enzymes are also capable of acting as catalysts for sialylation reactions due to their trans-sialidase activity and thus can be used in the context of the method of the present invention, preferably under kinetically controlled conditions.

Sialidases, which can be employed in the context of the present invention, can also comprise engineered sialidases. Based on sequence and structure comparisons, sialidase from Trypanosoma rangeli can be mutated at six positions, wherein the resulting mutant is able to display a significant level of trans-sialidase activity (see Paris et al. J. Mol. Biol. 345, 923 (2005)). On the other hand, truncation of a sialyl transferase form Photobacterium damsela resulted in an enzyme having α2-6-trans-sialidase activity (Cheng et al. Glycobiology 20, 260 (2010).

Even more preferably, the at least one enzyme having a sialidase and/or trans-sialidase activity can be selected from sialidases or trans-sialidases derived from Bifidobacterium longum subsp. infantis ATCC 15697, Bifidobacterium bifidum JCM1254, Bifidobacterium bifidum S17, Bifidobacterium bifidum PRL2010, Bifidobacterium bifidum NCIMB 41171, Trypanosoma cruzi, etc.

Even more preferably the at least one enzyme having a sialidase and/or trans-sialidase activity can be selected from sialidases or trans-sialidases as defined according to the following deposit numbers: gi|213524659 (Bifidobacterium longum subsp. infantis ATCC 15697), gi|213523006 Bifidobacterium longum subsp. infantis ATCC 15697), siab2 (Bifidobacterium bifidum JCM1254), further sialidases or trans-sialidases from Bifidobacterium bifidum JCM1254), gi|309252191 (Bifidobacterium bifidum S17), gi|309252190 (Bifidobacterium bifidum S17), gi|310867437 (Bifidobacterium bifidum PRL2010), gi|310867438 (Bifidobacterium bifidum PRL2010), gi|224283484 (Bifidobacterium bifidum NCIMB 41171), gi|313140638 (Bifidobacterium bifidum NCIMB 41171), gi|47252690 (Trypanosoma cruzi), gi|432485 (Trypanosoma cruzi), gi|343957998 (Trypanosoma congolense), gi|343958004 (Trypanosoma congolense), gi|2988379 (Photobacterium damsela) etc., or a sequence exhibiting a sequence identity with one of the above mentioned enzyme sequences having a sialidase and/or trans-sialidase activity of at least 70%, more preferably at least 80%, more preferably at least 85%, even more preferably at least 90% and most preferably at least 95% or even 97%, 98% or 99% as compared to the entire wild type sequence on amino acid level.

Additionally, wild type or engineered sialidases as defined above can be utilized herein, which display trans-sialidase activity and show a α2-3 and/or α2-6 regioselectivity. Such linkages are preferably targeted in the present invention. Such wild type or engineered sialidases preferably display trans-sialidase activity and catalyse the transfer of the sialyl residue to a galactosyl moiety of the polygalactosyl chain, preferably to a terminal galactosyl moiety of the polygalactosyl chain with 2-3 or 2-6 interglycosidic linkage.

Additionally, wild type or engineered sialidases as defined above, displaying transsialidase activity and showing a α2-3 and/or α2-6 regioselectivity, can catalyse the transfer of the sialyl residue to:
the galactose of the lacto-N-biosyl group with 2-3 interglycosidic linkage and/or
the N-acetyl-glucosamine of the lacto-N-biosyl group with 2-6 interglycosidic linkage and/or
the galactose of the N-acetyl-lactosaminyl group with 2-6 interglycosidic linkage and/or
a N-acetylglucosaminyl moiety, preferably to a terminal N-acetylglucosaminyl moiety with 2-3 or 2-6 interglycosidic linkage,
   provided that a lacto-N-biosyl, N-acetyl-lactosaminyl or N-acetylglucosaminyl group is already present in a modified GOS as a result of an antecedent enzymatic transfer of these glycosyl groups within the frame of the method of the present invention.

Also preferably, the regioselectivity the α-sialidases with sialidase/trans-sialidase activity used in the method of this invention matches the sialyl donor of formula 1 provided in step a) when X means a lactose moiety. In this regard, when 3′-SL is added in step a), then a α2-3-(trans)sialidase is preferably provided in step c), and when 6′-SL is added in step a), then a α2-6-(trans)sialidase is preferably provided in step c).

Particularly preferred sialidases or the wild types of engineered/truncated sialidases with sialidase/trans-sialidase activity are listed in the following Table 2:

TABLE 2
Preferred sialidases/trans-sialidases

| GI number in | |
|---|---|
| GenBank Database | Organisms |
| gi\|213524659 | Bifidobacterium longum subsp. infantis ATCC |
| | 15697 |
| gi\|213523006 | Bifidobacterium longum subsp. infantis ATCC |
| | 15697 |
| gi\|309252191 | Bifidobacterium bifidum S17 |
| gi\|309252190 | Bifidobacterium bifidum S17 |
| gi\|310867437 | Bifidobacterium bifidum PRL2010 |
| gi\|310867438 | Bifidobacterium bifidum PRL2010 |
| gi\|224283484 | Bifidobacterium bifidum NCIMB 41171 |
| gi\|313140638 | Bifidobacterium bifidum NCIMB 41171 |
| gi\|47252690 | Trypanosoma cruzi |
| gi\|432485 | Trypanosoma cruzi |
| gi\|343957998 | Trypanosoma congolense |
| gi\|343958004 | Trypanosoma congolense |
| gi\|2988379 | Photobacterium damsela |

Preferably, wild type or engineered sialidases display trans-sialidase activity and show a α2-6 regioselectivity. Such linkage is preferably targeted in the present invention. Such wild type or engineered sialidases preferably display trans-sialidase activity and catalyse the transfer of the sialyl residue to a galactosyl moiety of the polygalactosyl chain, preferably to a terminal galactosyl moiety of the polygalactosyl chain with 2-6 interglycosidic linkage. Even more preferably the enzyme displaying a trans-sialidase activity and having α2-6 regioselectivity can be selected from sialidases or trans-sialidases, or the wild types of engineered/truncated sialidases as defined according to the following deposit numbers: gi|213524659 Bifidobacterium longum subsp. infantis ATCC 15697, gi|213523006 Bifidobacterium longum subsp. infantis ATCC 15697, gi|309252191 Bifidobacterium bifidum S17, gi|309252190 Bifidobacterium bifidum S17, gi|310867437 Bifidobacterium bifidum PRL2010, gi|310867438 Bifidobacterium bifidum PRL2010, gi|224283484 Bifidobacterium bifidum NCIMB 41171, gi|313140638 Bifidobacterium bifidum NCIMB 41171, gi|2988379 Photobacterium damsela, etc.

Also preferably, wild type or engineered sialidases display trans-sialidase activity and show a α2-3 regioselectivity. Such linkage is preferably targeted in the present invention when 3′-SL (Neu5Acα2-3Galβ1-4Glc) is used as glycosyl donor. Such wild type or engineered sialidases preferably display trans-sialidase activity and catalyse the transfer of the sialyl residue to a galactosyl moiety of the polygalactosyl chain, preferably to a terminal galactosyl moiety of the polygalactosyl chain with 2-3 interglycosidic linkage. Even more preferably the enzyme displaying a trans-sialidase activity and having α2-3 regioselectivity can be selected from sialidases or trans-sialidases, or the wild types of engineered/truncated sialidases as defined according to the following deposit numbers: gi|213524659 Bifidobacterium longum subsp. infantis ATCC 15697, gi|213523006 Bifidobacterium longum subsp. infantis ATCC 15697, gi|309252191 Bifidobacterium bifidum S17, gi|309252190 Bifidobacterium bifidum S17, gi|310867437 Bifidobacterium bifidum PRL2010, gi|310867438 Bifidobacterium bifidum PRL2010, gi|224283484 Bifidobacterium bifidum NCIMB 41171, gi|313140638 Bifidobacterium bifidum NCIMB 41171, gi|47252690 Trypanosoma cruzi, gi|432485 Trypanosoma cruzi, gi|343957998 Trypanosoma congolense, gi|343958004 Trypanosoma congolense, etc.

Additionally, the at least one enzyme comprising a transglycosidase activity and/or a glycosynthase activity can be preferably selected from an enzyme exhibiting a lacto-N-biosidase or trans-lacto-N-biosidase activity, preferably as described in the following. In this context, enzymes having a lacto-N-biosidase or trans-lacto-N-biosidase activity are preferably selected from a lacto-N-biosidase or trans-lacto-N-biosidase as described in the following, e.g. lacto-N-biosidases (EC 3.2.1.140) as classified according to the GH20 family. Lacto-N-biosidases typically proceed through a retaining mechanism. Only two lacto-N-biosidases from Streptomyces and Bifidobacterium bifidum have been described and characterized up to now, which can be utilized in the present invention as a lacto-N-biosidase or trans-lacto-N-biosidase (see Sano et al. Proc. Natl. Acad. Sci. USA 89, 8512 (1992); Sano et al. J. Biol. Chem. 268, 18560 (1993); Wada et al. Appl. Environ. Microbiol. 74, 3996 (2008)). Lacto-N-biosidases specifically hydrolyse the terminal lacto-N-biosyl residue (β-D-Gal-(1→3)-D-GlcNAc) from the non-reducing end of oligosaccharides with the structure β-D-Gal-(1→3)-β-D-GlcNAc-(1→3)-β-D-Gal-(1→R). Wada et al. (supra) and Murata et al. (Glycoconj. J. 16, 189 (1999)) also demonstrated the ability of the lacto-N-biosidase from Bifidobacterium bifidum and Aureobacterium sp. L-101, respectively, to catalyse the transglycosylation by incubating donor substrates (such as lacto-N-tetraose and pNP-β-LNB) with acceptors (such as various 1-alkanols and lactose).

Even more preferably, the at least one enzyme having a lacto-N-biosidase or trans-lacto-N-biosidase activity can be selected from lacto-N-biosidases or trans-lacto-N-biosidases derived from Bifidobacterium bifidum JCM1254, Bifidobacterium bifidum PRL2010, Bifidobacterium bifidum NCIMB 41171, Aureobacterium sp. L-101 or Streptomyces sp., etc.

Even more preferably the at least one enzyme having a lacto-N-biosidase or trans-lacto-N-biosidase activity can be selected from lacto-N-biosidases or trans-lacto-N-biosidases as defined according to the following deposit numbers: gi|167369738 (Bifidobacterium bifidum JCM1254), gi|4096812 (Streptomyces sp.), gi|310867103 (Bifidobacterium bifidum PRL2010), gi|313140985 (Bifidobacterium bifidum NCIMB 41171), etc., or a sequence exhibiting a sequence identity with one of the above mentioned enzyme sequences having a lacto-N-biosidase or trans-lacto-N-biosidase activity of at least 70%, more preferably at least 80%, more preferably at least 85%, even more preferably at least 90% and most preferably at least 95% or even 97%, 98% or 99% as compared to the entire wild type sequence on amino acid level.

Furthermore, wild type or engineered lacto-N-biosidases as defined above can be utilized herein, which display trans-lacto-N-biosidase activity and show preferably a β1-3 regioselectivity. Such linkages are preferably targeted in the present invention. Such wild type or engineered lacto-N-biosidases preferably display trans-lacto-N-biosidase activity and catalyse the transfer of the lacto-N-biosyl residue to a galactosyl group with 1-3 interglycosidic linkage are targeted in the present invention.

Particularly preferred lacto-N-biosidases with lacto-N-biosidase or trans-lacto-N-biosidase activity are listed in the following Table 3:

TABLE 3
Preferred lacto-N-biosidases or trans-lacto-N-biosidases

| GI number in GenBank Database | Organisms |
|---|---|
| gi\|167369738 | Bifidobacterium bifidum JCM1254 |
| gi\|4096812 | Streptomyces sp. |
| gi\|310867103 | Bifidobacterium bifidum PRL2010 |
| gi\|313140985 | Bifidobacterium bifidum NCIMB |
| | 41171 |

Furthermore, the at least one enzyme comprising a transglycosidase activity and/or a glycosynthase activity can be preferably selected from an enzyme exhibiting a N-acetyllactosaminidase or trans-N-acetyllactosaminidase activity, preferably as described in the following. In this context, enzymes having a N-acetyllactosaminidase or trans-N-acetyllactosaminidase activity are preferably selected from a N-acetyllactosaminidase or trans-N-acetyllactosaminidase as described in the following, e.g. lacto-N-biosidases (EC 3.2.1.140) as classified according to the GH20 family. Particularly preferably, chitinase from bacillus circulans, more preferably chitinase A1 from Bacillus Circulans WL-12 as deposited under gi|142688, can be used as a N-acetyllactosaminidase or trans-N-acetyllactosaminidase, or a sequence exhibiting a sequence identity with one of the above mentioned enzyme sequences having a N-acetyllactosaminidase or trans-N-acetyllactosaminidase activity of at least 70%, more preferably at least 80%, equally more preferably at least 85%, even more preferably at least 90% and most preferably at least 95% or even 97%, 98% or 99% as compared to the entire wild type sequence on amino acid level. Notably, Shoda et al. showed that chitinase A1 from B. Circulans WL-12 is able to transfer N-acetyllactosamine with a β-1,6 glycosidic linkage using a 1,2-oxazoline derivative of N-acetyllactosamine (see Shoda et al. Cellulose 13, 477 (2006)).

Additionally, wild type or engineered glycosidases as defined above, which display trans-N-acetyllactosaminidase activity and show a β1-3 and/or β1-6 regioselectivity, can be used in the present invention. Such wild type or engineered glycosidases preferably display trans-N-acetyllactosaminidase activity and catalyse the transfer of the N-acetyl-lactosaminyl residue to a galactosyl group with 1-3 or 1-6 interglycosidic linkage.

Particularly preferred N-acetyllactosaminidases or trans-N-acetyllactosaminidases are listed in the following Table 4:

TABLE 4
Preferred N-acetyllactosaminidases or trans-N-acetyllactosaminidases

| GI number in the GenBank Database | Organisms |
|---|---|
| gi\|142688 | Bacillus circulans |

Furthermore, the at least one enzyme comprising a transglycosidase activity and/or a glycosynthase activity can be preferably selected from an enzyme exhibiting a N-acetylhexosaminidase or trans-N-acetylhexosaminidase activity, preferably as described in the following. In this context, enzymes having a N-acetylhexosaminidase or trans-N-acetylhexosaminidase activity are preferably selected from a N-acetylhexosaminidase or trans-N-acetylhexosaminidase as described in the following, e.g. β-N-acetylhexosaminidases (systematic name 2-acetamido-2-deoxy-β-D-hexopyranoside acetamidodeoxyhexohydrolases, EC 3.2.1.52 (exo-glycosidases) and 3.2.1.96 (endo-glycosidases)) as classified according to the GH3, GH18, GH20, GH56, GH84, GH85 and GH123 families. Among β-N-acetylhexosaminidases, β-N-acetylglucosaminidases are mainly found in GH3, GH18, GH20, GH84 and GH85 families.

β-N-Acetylhexosaminidases have been shown to be universally distributed among most types of living organisms, both prokaryotic and eukaryotic.

In vivo β-N-acetylhexosaminidases catalyse the hydrolysis of glycosidic linkages. When acting as exo-enzymes, they catalyse the cleavage of terminal β-D-GlcNAc andβ-D-GalNAc residues in N-acetyl-β-D-hexosaminides. In vitro they can catalyse the formation of a new glycosidic bond either by transglycosylation or by reverse hydrolysis (i.e. condensation, see review: Slamova et al., Biotechnology Advances 28, 682 (2010)).

Improved mutants displaying enhanced transglycosylation activity have been described previously. Mutants of endo-β-N-acetylglucosaminidase from Mucor hiemalis (Endo-M, GH85) as well as mutants of endo-N-acetylglucosaminidase from Streptococcus pneumoniae (Endo-D, GH85) exhibit glycosynthase-like activity using sugar oxazoline as a donor substrate (Umekawa et al. J. Biol. Chem. 283, 4469 (2008), Fan et al. ibid. 287, 11272 (2012)).

Additionally, wild type or engineered glycosidases as defined above can be utilized herein, which display trans-N-acetylglucosaminidase activity and show a β1-3 and/or β1-6 regioselectivity. Such wild type or engineered glycosidases preferably display trans-N-acetylglucosaminidase activity and catalyse the transfer of the N-acetyl-glucosaminyl residue to a galactosyl group with 1-3 or 1-6 interglycosidic linkage.

Particularly preferred N-acetylglucosaminidases or trans-N-acetylglucosaminidases are listed in the following Table 5, or a sequence exhibiting a sequence identity with one of the below mentioned enzyme sequences having a N-acetylglucosaminidase or trans-N-acetylglucosaminidase activity of at least 70%, more preferably at least 80%, more preferably at least 85%, even more preferably at least 90% and most preferably at least 95% or even 97%, 98% or 99% as compared to the entire wild type sequence on amino acid level.

TABLE 5
Preferred N-acetylglucosaminidases or trans-N-acetylglucosaminidases

| GI number in | |
|---|---|
| GenBank | |
| Database | Organisms |
| gi\|16077234 | Bacillus subtilis subsp. subtilis str. 168 |
| gi\|213522828 | Bifidobacterium longum subsp. infantis ATCC 15697 |
| gi\|213523089 | Bifidobacterium longum subsp. infantis ATCC 15697 |
| gi\|213524666 | Bifidobacterium longum subsp. infantis ATCC 15697 |
| gi\|292673292 | Bifidobacterium bifidum JCM 1254 |
| gi\|292673294 | Bifidobacterium bifidum JCM 1254 |
| gi\|4586325 | Lactobacillus casei ATCC 27092 |
| gi\|10518509 | Streptomyces plicatus |

As defined above, proteins comprising a transglycosidase and/or a glycosynthase activity as defined above can also comprise engineered proteins comprising a transglycosidase and/or a glycosynthase activity. It is particularly envisaged that wild type or mutated glycosidases displaying a transfucosidase, transsialidase, trans-N-acetylglucosaminidase, trans-lacto-N-biosidase and/or trans-N-acetyllactosaminidase activity, preferably a α-transfucosidase, α-transsialidase, β-trans-N-acetylglucosaminidase, β-trans-lacto-N-biosidase and/or β-trans-N-acetyllactosaminidase activity, can be used in the present invention to produce such oligosaccharides. Preparation of such enzymes is preferably carried out via site directed mutagenesis approaches or directed evolution.

In rational engineering, novel altered enzymes (mutants) are created via site directed mutagenesis approaches, preferably by introduction of point mutations. This technique generally requires reliance on the static 3D protein structure. The mutations generally affect the active site of the enzymes such that they lose their ability to degrade their transglycosylation products but remain capable of synthesis. A preferred strategy consists of the replacement of the catalytic nucleophile by a non-nucleophilic residue. This modification results in the formation of an inactive mutant or an altered enzyme with reduced transglycosylation activity due the lack of appropriate environment for the formation of the reactive host-guest complex for transglycosylation. However, in the presence of a more active glycosyl donor (e.g. glycosyl fluoride) that mimics the glycosyl enzyme intermediate, the mutated enzyme is able to transfer efficiently the glycosyl moiety to a suitable acceptor generating a glycoside with inverted anomeric stereochemistry. Such a mutant glycosidase is termed a glycosynthase and their development represents one of the major advances in the use of glycosidases for synthetic purposes. In principle, the glycosynthase concept can be applied to all GH specificities and offer a large panel of enzymes potentially able to synthesize various oligosaccharides with very high yields, up to 95%.

The second preferred technique is called directed evolution. This strategy comprises random mutagenesis applied to the gene of the selected glycosidase, which thus generates a library of genetically diverse genes expressing glycosidase. Generation of sequence diversity can be performed using well-known methodologies, the most preferable being the error prone polymerase chain reaction (epCR) method. This gene library can be inserted into suitable microorganisms such as E. coli or S. cerevisiae for producing recombinant variants with slightly altered properties. Clones expressing improved enzymes are then identified with a fast and reliable screening method, selected and brought into a next round of mutation process. The recursive cycles of mutation, recombination and selection are continued as far as mutant(s) with the desired activity and/or specificity is/are evolved. To date, different high-throughput screening methodologies for glycosidases including glycosynthases have been developed. Applying these approaches, effective engineered transglycosidases, including new and more efficient glycosynthases can and have been created and isolated. An α-L-fucosidase from Thermotoga maritima has been recently converted into an efficient α-L-transfucosidase by directed evolution. The transferase/hydrolysis ratio of the evolved enzyme was 30 times higher than the native enzyme (see Osanjo et al. above).

Proteins comprising a transglycosidase and/or a glycosynthase activity as defined above can also comprise fragments or variants of those protein sequences. Such fragments or variants can typically comprise a sequence having a sequence identity with one of the above mentioned protein sequences of at least 70%, more preferably at least 80%, equally more preferably at least 85%, even more preferably at least 90% and most preferably at least 95% or even 97%, 98% or 99% as compared to the entire wild type sequence on amino acid level.

“Fragments” of proteins or peptides in the context of the present invention can also comprise a sequence of a protein or peptide as defined herein, which is, with regard to its amino acid sequence N-terminally, C-terminally and/or intrasequentially truncated compared to the amino acid sequence of the original (native) protein. Such truncation can thus occur either on the amino acid level or correspondingly on the nucleic acid level. A sequence identity with respect to such a fragment as defined herein can therefore preferably refer to the entire protein or peptide as defined herein or to the entire (coding) nucleic acid molecule of such a protein or peptide. Likewise, “fragments” of nucleic acids in the context of the present invention can comprise a sequence of a nucleic acid as defined herein, which is, with regard to its nucleic acid molecule 5′-, 3′- and/or intrasequentially truncated compared to the nucleic acid molecule of the original (native) nucleic acid molecule. A sequence identity with respect to such a fragment as defined herein can therefore preferably refer to the entire nucleic acid as defined herein.

“Variants” of proteins or peptides as defined in the context of the present invention (e.g. as encoded by a nucleic acid as defined herein) can be encoded by the nucleic acid molecule of a polymeric carrier cargo complex. Thereby, a protein or peptide can be generated, having an amino acid sequence which differs from the original sequence in one or more mutation(s), such as one or more substituted, inserted and/or deleted amino acid(s). Preferably, these fragments and/or variants have the same biological function or specific activity compared to the full-length native protein, e.g. its specific antigenic property.

“Variants” of proteins or peptides as defined in the context of the present invention (e.g. as encoded by a nucleic acid as defined herein) can also comprise conservative amino acid substitution(s) compared to their native, i.e. non-mutated physiological, sequence. Those amino acid sequences as well as their encoding nucleotide sequences in particular fall under the term variants as defined herein. Substitutions in which amino acids that originate from the same class are exchanged for one another are called conservative substitutions. In particular, these are amino acids having aliphatic side chains, positively or negatively charged side chains, aromatic groups in the side chains or amino acids having side chains that can enter into hydrogen bridges, e.g. side chains which have a hydroxyl function. This means that e.g. an amino acid having a polar side chain is replaced by another amino acid having a likewise polar side chain, or, for example, an amino acid characterized by a hydrophobic side chain is substituted by another amino acid having a likewise hydrophobic side chain (e.g. serine (threonine) by threonine (serine) or leucine (isoleucine) by isoleucine (leucine)). Insertions and substitutions are possible, in particular, at those sequence positions which cause no modification to the three-dimensional structure or do not affect the binding region. Modifications to a three-dimensional structure by insertion(s) or deletion(s) can easily be determined e.g. using CD spectra (circular dichroism spectra) (Urry, 1985, Absorption, Circular Dichroism and ORD of Polypeptides, in: Modern Physical Methods in Biochemistry, Neuberger et al. (ed.), Elsevier, Amsterdam).

Furthermore, variants of proteins or peptides as defined herein can also comprise those sequences wherein nucleotides of the nucleic acid are exchanged according to the degeneration of the genetic code, without leading to an alteration of the respective amino acid sequence of the protein or peptide, i.e. the amino acid sequence or at least part thereof can not differ from the original sequence in one or more mutation(s) within the above meaning.

In order to determine the percentage to which two sequences are identical, e.g. nucleic acid sequences or amino acid sequences as defined herein, preferably the amino acid sequences encoded by a nucleic acid sequence of the polymeric carrier as defined herein or the amino acid sequences themselves, the sequences can be aligned in order to be subsequently compared to one another. Therefore, e.g. a position of a first sequence can be compared with the corresponding position of the second sequence. If a position in the first sequence is occupied by the same component as is the case at a position in the second sequence, the two sequences are identical at this position. If this is not the case, the sequences differ at this position. If insertions occur in the second sequence in comparison to the first sequence, gaps can be inserted into the first sequence to allow a further alignment. If deletions occur in the second sequence in comparison to the first sequence, gaps can be inserted into the second sequence to allow a further alignment. The percentage to which two sequences are identical is then a function of the number of identical positions divided by the total number of positions including those positions which are only occupied in one sequence. The percentage to which two sequences are identical can be determined using a mathematical algorithm. A preferred, but not limiting, example of a mathematical algorithm which can be used is the algorithm of Karlin et al. PNAS USA 90, 5873 (1993) or Altschul et al. Nucleic Acids Res. 25, 3389 (1997). Such an algorithm is integrated in the BLAST program. Sequences which are identical to the sequences of the present invention to a certain extent can be identified by this program.

The proteins as added in step c) can be provided in a free form or alternatively be bound to or immobilized onto a surface. In this specific case, the order of steps a), b) and c) is preferably inverted. Binding to or immobilization onto a surface can be carried out e.g. via electrostatic bonds, van der Waals-bonds, covalent bonds, etc. Binding to or immobilization onto a surface can be furthermore carried out using a covalent linker or a crosslinker, or a Tag, as known to a skilled person for purification of proteins. Such tags comprise, inter alia, affinity tags or chromatography tags. Affinity tags can include e.g. chitin binding protein (CBP), maltose binding protein (MBP), glutathione-S-transferase (GST), or the Strep-Tag. The poly(His) tag is a widely-used protein tag that binds to metal matrices. Chromatography tags are used to alter chromatographic properties of the protein to afford different resolution across a particular separation technique, and include e.g. polyanionic amino acids based tags, such as the FLAG-tag. The surface can be the surface of a bioreactor, or any suitable reaction chamber.

According to step d) a mixture is prepared from substances provided by steps a), b) and c). Preferably, such a mixture according to step d) represents a mixture of one, two, three, four, five, one to five, three to ten, five to ten or even more different donors as defined according to step a), and one, two, three, four, five, two to five, two to ten, two to twenty, five to ten or even more different enzymes comprising transglycosidase activity and/or glycosynthase activity.

In a further step e) the mixture containing at least one compound as defined according to step a), at least one compound as defined according to step b), and at least one enzyme as added according to step c), together forming a mixture according to step d), are incubated to allow generation of modified GOS via enzymatic means using the at least one enzyme comprising a transglycosidase activity and/or a glycosynthase activity as defined herein. Such an incubation advantageously allows the generation of a multiplicity of different modified GOS.

Generation of such a multiplicity of different modified GOS is based on the use of enzymes with different activities provided in step c), but also on the use of diverse donors and acceptors according to steps a) and b), preferably as a mixture as already outlined in step d). Utilizing this approach, the method of the present invention advantageously allows variation of the possible number and type of oligosaccharides obtainable by the method in a simple and cost efficient manner. The use of enzymes furthermore allows the preparation of various modified GOS to be carried out in a stereoselective manner. Generation of modified GOS preferably occurs by transferring glycosyl moieties (e.g., a sialyl moiety, fucosyl moiety, N-acetylglucosaminyl moiety, N-acetyllactosaminyl moiety, or lacto-N-biosyl moiety) by forming new bonds at desired positions of the molecule, etc., in a well defined manner to obtain a mixture of various modified GOS.

Incubation according to step e) preferably occurs with a concentration of (each of the) enzymes in a concentration of 1 mU/l to 1,000 U/l, preferably 10 mU/l to 100 U/l, when the activity capable of forming 1 μmol of specific product for a defined protein starting from a defined educt is defined as 1 unit (U), e.g. for a glycotransferase the production of a glycose-containing complex carbohydrate at 37° C. in 1 minute. The activity of each enzyme as defined herein can be assessed with respect to its naturally occurring or engineered substrate.

The incubation according to step e) can be carried out in a reaction medium, preferably an aqueous medium, comprising the mixture obtained according to step d) of the method of the present invention and optionally water; a buffer such as a phosphate buffer, a carbonate buffer, an acetate buffer, a borate buffer, a citrate buffer and a tris buffer, or combinations thereof; alcohol, such as methanol and ethanol; ester such as ethyl acetate; ketone such as acetone; amide such as acetamide; and the like.

Furthermore, the incubation according to step e) can be carried out in a reaction medium as defined above, wherein optionally a surfactant or an organic solvent can be added, if necessary. Any surfactant capable of accelerating the formation of a complex carbohydrate as defined according to the present invention as a possible product of the invention can be used as the surfactant. Examples include non-ionic surfactants such as polyoxyethylene octadecylamine (e.g., Nymeen S-215, manufactured by Nippon Oil & Fats); cationic surfactants, such as cetyltrimethylammonium bromide and alkyldimethyl benzylammoniumchloride (e.g., Cation F2-40E, manufactured by Nippon Oil & Fats); anionic surfactants such as lauroyl sarcosinate; tertiary amines such as alkyldimethylamine (e.g., Tertiary Amine FB, manufactured by Nippon Oil & Fats); and the like, which are used alone or as a mixture of two or more. The surfactant can be used generally in a concentration of 0.1 to 50 g/l. The organic solvent can include xylene, toluene, fatty acid alcohol, acetone, ethyl acetate, and the like, which can be used in a concentration of generally 0.1 to 50 ml/l.

The incubation according to step e) can be furthermore carried out in a reaction medium as defined above, preferably having a pH 3 to 10, pH 5 to 10, preferably pH 6 to 8.

The incubation according to step e) can be furthermore carried out at a temperature of about 0° C. to about 100° C., preferably at a temperature of about 10 to about 50° C., e.g. at a temperature of about 20° C. to about 50° C. In case of using enzymes stemming from thermophilic organisms (e.g. Thermotoga sp.) even higher temperature is beneficial, such as about 50 to about 80° C., preferably at about 60° C. In the reaction medium, inorganic salts, such as MnCl₂ and MgCl₂, can be added, if necessary.

The incubation according to step e) of the method of the present invention can be carried out in a bioreactor. The bioreactor is preferably suitable for either a continuous mode or a discontinuous mode.

The incubation according to step e) can be carried out in a continuous or discontinuous mode. If carried out in a continuous mode, the method preferably provides for a continuous flow of compounds and/or enzymes as necessary, preferably by continuously providing educts of the reaction to the reaction mixture and continuously removing products from the reaction mixture, while maintaining the concentration of all components, including enzymes at a predetermined level. The enzymes used in a continuous mode can be added either in free form or as bound or immobilized to a surface.

With regard to a certain embodiment of the first aspect, the addition of donors and/or enzymes can be repeated according to an optional step f). Particularly preferably, steps a), c), d) and e) can be repeated, preferably with the mixture obtained according to step e). When repeating at least one of the steps a), c), d) and e), the at least one compound provided according to step a) is preferably different from that/those provided in the previous cycle, and the at least one enzyme provided according to step c) is preferably different from that/those provided in the previous cycle. Such a stepwise proceeding can allow within multiple rounds the rational generation of a defined set of compounds in a controllable manner. It can also provide for a rational exclusion of specific components. To obtain such a variety of compounds, the compounds and/or enzymes can be added simultaneously or sequentially, and preferably compounds and/or enzymes can be added simultaneously in one step and/or sequentially in different steps.

Alternatively, a mixture with at least one compound as defined herein for step a) and at least one compound as defined herein for step b) and at least one enzyme as defined herein according to step c) can be incubated in one step without repetition of one or more steps.

Such a proceeding can be preferable in certain circumstances, as it can lead to the largest variety of possible products.

The method of the present invention as defined above preferably leads to generation of compounds on the basis of donors as provided in step a) and acceptors as provided in step b) upon adding enzymes according to step c) and incubating the mixture (step e)). An optional repetition of these steps can be carried out as defined above. Preferably, according to the second aspect of the invention, the method as described above results in either a single modified galactooligosaccharide or a diversified mixture comprising two or more modified GOS, the single compounds of which can be defined as a galactooligosaccharide comprising at least one glycosyl residue, said glycosyl residue, being different from galactosyl, is coupled, by its anomeric carbon atom, to any of the monosaccharide units of a galactooligosaccharide represented by the formula (Gal)ₙ-A, wherein A means galactose or glucose, preferably glucose, and n is at least 2.

Also preferably, the method as described above results in either a single modified galactooligosaccharide or a diversified mixture comprising two or more modified GOS, the single compounds of which can be defined as a galactooligosaccharide comprising at least one glycosyl residue, said glycosyl residue, being different from galactosyl and α2-3-sialyl, is coupled, by its anomeric carbon atom, to any of the monosaccharide units of a galactooligosaccharide represented by the formula (Gal)ₙ-A, wherein A means galactose or glucose, preferably glucose, and n is at least 2.

According to a preferred embodiment, the (Gal)ₙ moiety represents a linear or branched polygalactopyranosyl chain wherein the galactopyranosyl units can be coupled to each other by β1-2 and/or β1-3 and/or β1-4 and/or β1-6 interglycosidic linkages, preferably β1-3 and/or β1-4 and/or β1-6 linkages. Also preferably, n ranges between 2 to 15, more preferably 2 to 10, even more preferably 2 to 6. When A means glucose, the galactose unit is linked to it preferably by β1-4 interglycosidic linkage thus forming a lactose unit at the reducing end. Most important galactooligosaccharide core structures are: Galpβ1-6Galpβ1-4Glc, Galpβ1-3Galpβ1-4Glc, Galpβ1-4Galpβ1-4Glc, Galpβ1-4Galpβ1-6Glc, Galpβ1-4Galpβ1-3Glc, Galpβ1-4Galpβ1-2Glc, Galpβ1-6Galpβ1-6Gal, Galpβ1-4Galpβ1-4Galpβ1-4Glc, Galpβ1-6Galpβ1-6Galpβ1-4Glc, Galpβ1-3Galpβ1-6Galpβ1-4Glc, Galpβ1-6Galpβ1-3Galpβ1-4Glc, Galpβ1-3Galpβ1-3Galpβ1-4Glc, Galpβ1-6Galpβ1-6Galpβ1-6Galpβ1-4Glc, Galpβ1-3Galpβ1-3Galpβ1-3Galpβ1-4Glc, Galpβ1-3Galpβ1-3Galpβ1-3Galpβ1-3Galpβ1-4Glc, Galβ1-3Galpβ1-3Galpβ1-3Galpβ1-3Galpβ1-3Galpβ1-4Glc.

Also in a preferred case, the at least one glycosyl residue comprised by the modified galactooligosaccharide according to the present invention can be linked to any of the monosaccharide units of the galactooligosaccharide core, that is to any of the galactosyl units of the (Gal)ₙ chain and/or to residue A being glucose or galactose. More preferably, said at least one glycosyl moiety can be a monosaccharide unit such as sialyl, fucosyl and/or N-acetylglucosaminyl, or a disaccharide unit such as N-acetyllactosaminyl, lacto-N-biosyl or sialylated and/or fucosylated N-acetylglucosaminyl, or tri-, tetra- or polysaccharide unit such as N-acetyllactosaminyl and lacto-N-biosyl moieties further glycosylated with sialyl and/or fucosyl and/or N-acetyllactosaminyl and/or lacto-N-biosyl and/or N-acetylglucosaminyl.

Yet preferably, the at least one glycosyl residue comprised by the modified galactooligosaccharide according to the present invention can be linked to any of the monosaccharide units of the galactooligosaccharide core, that is to any of the galactosyl units of the (Gal)ₙ chain and/or to residue A being glucose or galactose. More preferably, said at least one glycosyl moiety can be a monosaccharide unit such as sialyl (expect for α2-3-sialyl), fucosyl and/or N-acetylglucosaminyl, or a disaccharide unit such as N-acetyllactosaminyl, lacto-N-biosyl or sialylated and/or fucosylated N-acetylglucosaminyl, or tri-, tetra- or polysaccharide unit such as N-acetyllactosaminyl and lacto-N-biosyl moieties further glycosylated with sialyl and/or fucosyl and/or N-acetyllactosaminyl and/or lacto-N-biosyl and/or N-acetylglucosaminyl.

Particularly preferably, compounds obtained according to the method of the present invention as defined above are characterized by their linkages and modifications. Preferably, the compounds obtained by the method of the present invention after incubation step e) and/or a repetition of steps according to step f) are characterized in that one or more of the following are present:
a N-acetyl-glucosaminyl group is attached to a galactosyl moiety of the polygalactosyl chain, preferably to a terminal galactosyl moiety of the polygalactosyl chain with 1-3, 1-4 or 1-6 interglycosidic linkage, preferably with 1-3 or 1-6 linkage,
a N-acetyl-glucosaminyl group is attached to the galactosyl moiety of a N-acetyl-lactosaminyl or lacto-N-biosyl group with 1-3 or 1-6 interglycosidic linkage,
a N-acetyl-lactosaminyl group is attached to the galactosyl moiety of another N-acetyl-lactosaminyl group with 1-3 or 1-6 interglycosidic linkage,
a N-acetyl-lactosaminyl group is attached to the galactosyl moiety of a lacto-N-biosyl group with 1-3 or 1-6 interglycosidic linkage,
a N-acetyl-lactosaminyl group is attached to a galactosyl moiety of the polygalactosyl chain, preferably to a terminal galactosyl moiety of the polygalactosyl chain with 1-3, 1-4 or 1-6 interglycosidic linkage, preferably with 1-3 or 1-6 linkage,
a lacto-N-biosyl group is attached to the galactosyl moiety of a N-acetyl-lactosaminyl group with 1-3 or 1-6 interglycosidic linkage, preferably with 1-3 linkage,
a lacto-N-biosyl group is attached to the galactosyl moiety of another lacto-N-biosyl group with 1-3 or 1-6 interglycosidic linkage, preferably with 1-3 linkage,
a lacto-N-biosyl group is attached to a galactosyl moiety of the polygalactosyl chain, preferably to a terminal galactosyl moiety of the polygalactosyl chain with 1-3, 1-4 or 1-6 interglycosidic linkage, preferably with 1-3 or 1-6 linkage,
a fucosyl residue attached to a N-acetyl-lactosaminyl and/or a lacto-N-biosyl group is linked to
   the galactose of the lacto-N-biosyl group with 1-2 interglycosidic linkage and/or
   the N-acetyl-glucosamine of the lacto-N-biosyl group with 1-4 interglycosidic linkage and/or
   the N-acetyl-glucosamine of the N-acetyl-lactosaminyl group with 1-3 interglycosidic linkage,
a fucosyl residue is attached to a galactosyl moiety of the polygalactosyl chain, with 1-2 or 1-3 interglycosidic linkage,
a fucosyl residue is attached to the galactose or glucose of moiety A with 1-2 or 1-3 interglycosidic linkage,
a fucosyl residue is attached to a N-acetylglucosaminyl moiety, preferably to a terminal N-acetylglucosaminyl moiety with 1-3 or 1-4 interglycosidic linkage,
a sialyl residue attached to the N-acetyl-lactosaminyl and/or the lacto-N-biosyl group is linked to
   the galactose of the lacto-N-biosyl group with 2-3 interglycosidic linkage and/or
   the N-acetyl-glucosamine of the lacto-N-biosyl group with 2-6 interglycosidic linkage and/or
   the galactose of the N-acetyl-lactosaminyl group with 2-6 interglycosidic linkage,
a sialyl residue is attached to a galactosyl moiety of the polygalactosyl chain, preferably to a terminal galactosyl moiety of the polygalactosyl chain with 2-3 or 2-6 interglycosidic linkage,
a sialyl residue is attached to a N-acetylglucosaminyl moiety, preferably to a terminal N-acetylglucosaminyl moiety with 2-3 or 2-6 interglycosidic linkage.

According to a further preferred aspect, the interglycosidic linkages in the compounds of the invention are β, with the exception of fucosyl and sialyl residues which are attached via α-linkage.

Yet preferably, the at least one glycosyl residue comprised by the modified galactooligosaccharide according to the present invention is a sialyl group. This sialyl group is preferably attached to a galactosyl moiety of the polygalactosyl chain, more preferably to a terminal galactosyl moiety of the polygalactosyl chain with α2-3 interglycosidic linkage when 3′-SL is used as sialyl donor. The sialylated galactooligosaccharide of this type can carry more than one sialyl group.

Also preferably, in a sialylated galactooligosaccharide obtained or obtainable of the method according to the present invention, the sialyl group is attached to a galactosyl moiety of the polygalactosyl chain, more preferably to a terminal galactosyl moiety of the polygalactosyl chain with α2-6 interglycosidic linkage. The sialylated galactooligosaccharide of this type can carry more than one sialyl group. According to a further preferred embodiment, the at least one glycosyl residue comprised by the modified galactooligosaccharide according to the present invention is a fucosyl group. The fucosyl residue is attached to a galactosyl moiety of the polygalactosyl chain or to the galactose or glucose of moiety A, with 1-2 or 1-3 interglycosidic linkage, preferably with a α1-2 interglycosidic linkage. The fucosylated galactooligosaccharide of this type can carry more than one fucosyl group.

According to a further embodiment of the second aspect, the present invention provides a compound, preferably a mixture of compounds obtained or obtainable by the method of the present invention as described herein. In this context, such a mixture of compounds obtained or obtainable by the method of the present invention as described herein is preferably to be understood as a mixture of at least 2 to 10, 2 to 20, 2 to 100, 2 to 200, or even more different compounds as generally defined above. Such compounds can be preferably selected without restriction from any of the compounds as defined above.

The present invention also provides or utilizes salts of herein defined compounds. Such salts can be preferably selected from salts of the compounds defined above, which contain at least one sialyl residue: an associated ion pair consisting of the negatively charged acid residue of sialylated GOS and one or more cations in any stoichiometric proportion. Cations, as used in the present context, are atoms or molecules with positive charge. The cation can be inorganic or organic. Preferred inorganic cations are ammonium ion, alkali metal, alkali earth metal and transition metal ions, more preferably Na⁺, Ca²⁺, Mg²⁺, Ba²⁺, Fe²⁺, Zn²⁺, Mn²⁺ and Cu²⁺, most preferably K′, Ca²⁺, Mg²⁺, Ba²⁺, Fe²⁺ and Zn²⁺. Basic organic compounds in positively charged form can be relevant organic cations. Such preferred positively charged counterparts are diethyl amine, triethyl amine, diisopropyl ethyl amine, ethanolamine, diethanolamine, triethanolamine, imidazole, piperidine, piperazine, morpholine, benzyl amine, ethylene diamine, meglumin, pyrrolidine, choline, tris-(hydroxymethyl)-methyl amine, N-(2-hydroxyethyl)-pyrrolidine, N-(2-hydroxyethyl)-piperidine, N-(2-hydroxyethyl)-piperazine, N-(2-hydroxyethyl)-morpholine, L-arginine, L-lysine, oligopeptides having L-arginine or L-lysine unit or oligopeptides having a free amino group on the N-terminal, etc., all in protonated form. Such salt formations can be used to modify characteristics of the complex molecule as a whole, such as stability, compatibility to excipients, solubility and the ability to form crystals.

According to the third aspect, compounds or a mixture of compounds obtained or obtainable by the method of the present invention as described herein have, preferably enhanced, bifidogenic activity compared to the precursor GOS. The compounds described above are particularly effective in the improvement and maturation of the immune system of neonatal infants, and has preventive effect against secondary infections following viral infections such as influenza. The use of compounds or a mixture of compounds obtained or obtainable by the method of the present invention as described herein as prebiotic enhances the beneficial effects and efficiency of probiotics, such as Lactobacillus and Bifidobacterium species, in promoting the development of an early bifidogenic intestinal microbiota in infants, in reducing the risk of development or allergy and/or asthma in infants, and in preventing and treating pathogenic infections in such as diarrhoea in infants.

According to the fourth aspect, the present invention provides compounds or a mixture of compounds obtained or obtainable by the method of the present invention as described herein for use in enhancing the bifidogenic effect of consumable products. The compounds described above are particularly effective in the improvement and maturation of the immune system of neonatal infants, and has preventive effect against secondary infections following viral infections such as influenza. The use of compounds or a mixture of compounds obtained or obtainable by the method of the present invention as described herein as prebiotics enhances the beneficial effects and efficiency of probiotics, such as Lactobacillus and Bifidobacterium species, in promoting the development of an early bifidogenic intestinal microbiota in infants, in reducing the risk of development or allergy and/or asthma in infants, and in preventing and treating pathogenic infections such as diarrhoea in infants.

According to the fifth aspect, compounds or a mixture of compounds obtained or obtainable by the method of the present invention as described herein can be used for the preparation of a consumable product, preferably for the preparation of a pharmaceutical composition, a nutritional formulation or a food supplement. Such a compound or mixture of compounds obtained or obtainable by the method of the present invention as described herein is particularly effective in the improvement and maturation of the immune system of neonatal infants, and has preventive effect against secondary infections following viral infections such as influenza. The use of compounds or a mixture of compounds obtained or obtainable by the method of the present invention as described herein as prebiotic enhances the beneficial effects and efficiency of probiotics, such as Lactobacillus and Bifidobacterium species, in promoting the development of an early bifidogenic intestinal microbiota in infants, in reducing the risk of development or allergy and/or asthma in infants, and in preventing and treating pathogenic infections such as diarrhoea in infants.

In an embodiment, the present invention provides a pharmaceutical composition comprising compounds or a mixture of compounds obtained or obtainable by the method of the present invention, the single compounds of which can be defined as a galactooligosaccharide comprising at least one glycosyl residue, said glycosyl residue, being different from galactosyl and α2-3-sialyl, is coupled, by its anomeric carbon atom, to any of the monosaccharide units of a galactooligosaccharide represented by the formula (Gal)ₙ-A, wherein A means galactose or glucose, preferably glucose, and n is at least 2, and preferably a pharmaceutically acceptable carrier. “Pharmaceutically acceptable carriers” include but are not limited to additives, adjuvants, excipients and diluents (water, gelatine, talc, sugars, starch, gum arabic, vegetable gums, vegetable oils, polyalkylene glycols, flavouring agents, preservatives, stabilizers, emulsifying agents, lubricants, colorants, fillers, wetting agents, etc.). Suitable carriers are described in the most recent edition of Remington's Pharmaceutical Sciences, a standard reference text in the field. The dosage form for administration includes, for example, tablets, powders, granules, pills, suspensions, emulsions, infusions, capsules, injections, liquids, elixirs, extracts and tinctures.

In another embodiment, nutritional formulations are provided such as foods or drinks, comprising compounds or a mixture of compounds obtained or obtainable by the method of the present invention as described herein. The nutritional formulation can contain edible micronutrients, vitamins and minerals as well. The amounts of such ingredient can vary depending on whether the formulation is intended for use with normal, healthy infants, children, adults or subjects having specialized needs (e.g. suffering from metabolic disorders). Micronutrients include, for example, edible oils, fats or fatty acids (such as coconut oil, soy-bean oil, monoglycerides, diglycerides, palm olein, sunflower oil, fish oil, linoleic acid, linolenic acid etc.), carbohydrates (such as glucose, fructose, sucrose, maltodextrin, starch, hydrolysed corn-starch, etc.) and proteins from casein, soy-bean, whey or skim milk, or hydrolysates of these proteins, but protein from other sources (either intact or hydrolysed) can be used. Vitamins can be chosen from the group consisting of vitamin A, B1, B2, B5, B6, B12, C, D, E, H, K, folic acid, inositol and nicotinic acid. The nutritional formula can contain the following minerals and trace elements: Ca, P, K, Na, Cl, Mg, Mn, Fe, Cu, Zn, Se, Cr or I.

According to a general embodiment of the fifth aspect, a nutritional formulation as defined above can further contain one or more probiotics, e.g. lacto bacteriae, Bifidobacterium species, prebiotics such as fructooligosaccharides and galactooligosaccharides, proteins from casein, soy-bean, whey or skim milk, carbohydrates such as lactose, saccharose, maltodextrin, starch or mixtures thereof, lipids (e.g. palm olein, sunflower oil, safflower oil) and vitamins and minerals essential in a daily diet. Probiotics are preferably also contained in the nutritional formulation in an amount sufficient to achieve the desired effect in an individual, preferably in infants, children and/or adults.

In a preferred embodiment, the nutritional formulation as defined above is an infant formula. In the context of the present invention, the term “infant formula” preferably means a foodstuff intended for particular nutritional use by infants during the first 4-6 months or even 4 to 12 months of life and satisfying by itself the nutritional requirements of infants. It can contain one or more probiotic Bifidobacterium species, prebiotics such as fructooligosaccharides and galactooligosaccharides, proteins from casein, soy-bean, whey or skim milk, carbohydrates such as lactose, saccharose, maltodextrin, starch or mixtures thereof, lipids (e.g. palm olein, sunflower oil, safflower oil) and vitamins and minerals essential in a daily diet.

In an embodiment, a food supplement can be provided. Such a food supplement contains ingredients as defined for nutritional food above, e.g. compounds or a mixture of compounds obtained or obtainable by the method of the present invention as described herein, vitamins, minerals, trace elements and other micronutrients, etc. The food supplement can be for example in the form of tablets, capsules, pastilles or a liquid. The supplement can contain conventional additives selected from but not limited to binders, coatings, emulsifiers, solubilising agents, encapsulating agents, film forming agents, adsorbents, carriers, fillers, dispersing agents, wetting agents, gellifying agents, gel forming agents, etc.

According to a preferred embodiment, the food supplement is a digestive health functional food, as the administration of compounds or a mixture of compounds obtained or obtainable by the method of the present invention as described herein provides a beneficial effect on digestive health. A digestive health functional food is preferably a processed food used with the intention of enhancing and preserving digestive health by utilizing compounds or a mixture of compounds obtained or obtainable by the method of the present invention as described herein, as physiologically functional ingredients or components in the form of a tablet, capsule, powder, etc. Different terms such as dietary supplement, nutraceutical, designed food, or health product can also be used to refer to a digestive health functional food.

In a further embodiment, compounds or a mixture of compounds obtained or obtainable by the method of the present invention as described herein, can be used for the preparation of nutritional formulations including foods, drinks and feeds, preferably infant formulae, food supplements and digestive health functional foods, preferably any of these as described above. The nutritional formulation can be prepared in any usual manner.

According to the seventh aspect, the present invention relates to a method for enhancing the bifidogenic effect of galactooligosaccharides, characterized in that at least one glycosyl donor is reacted with a precursor galactooligosaccharide represented by the formula (Gal)ₙ-A or a mixture thereof, wherein A and n are as defined above, under the catalysis of an enzyme capable of transferring said glycosyl moiety to said precursor galactooligosaccharide, provided that the glycosyl group is not galactosyl.

This method can be suitably carried out in the same way as the method of the first aspect of the invention as described above.

Accordingly, the at least one glycosyl donor is preferably selected from the group consisting of a sialyl donor, a fucosyl donor, and an optionally galactosylated N-acetyl-glucosaminyl donor.

Preferably, the sialyl and/or fucosyl and/or optionally galactosylated N-acetyl-glucosaminyl donor has a leaving group selected from the group consisting of fluoro, azido and —OR group, wherein R can be a mono-, di- or oligosaccharide, glycolipid, glycoprotein or glycopeptide, cyclic or acyclic aliphatic group, or aryl residue, or wherein the optionally galactosylated N-acetyl-glucosaminyl donor is an oxazoline.

More preferably, the sialyl donor is characterized by formula 1, and/or the fucosyl donor is characterized by formula 2, and/or the optionally galactosylated N-acetyl-glucosaminyl donor is characterized by formulae 3 or 4

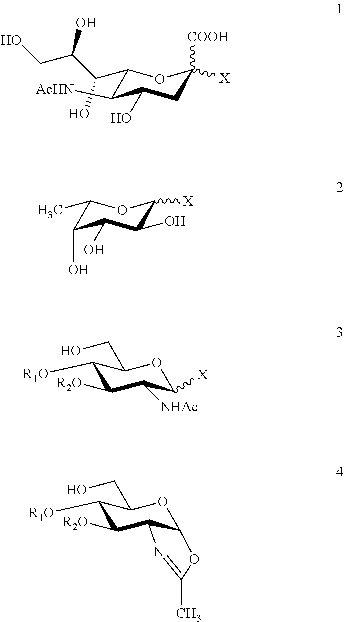

wherein X, independently, is selected from the group consisting of azide, fluoro, optionally substituted phenoxy, optionally substituted pyridinyloxy, lactose moiety, group A, group B, group C and group D

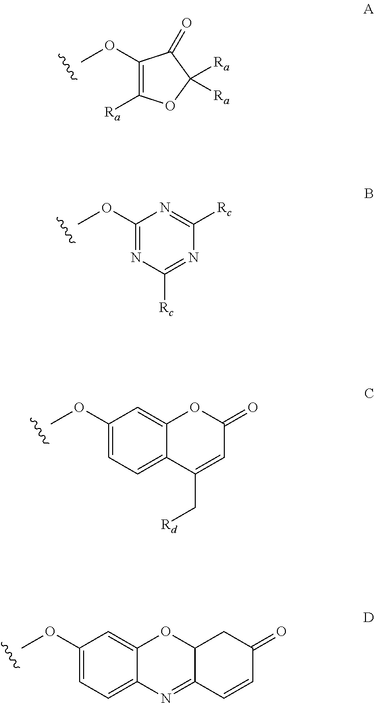

wherein Rₐ is independently H or alkyl, or two vicinal Rₐ groups represent a ═C(R_{b})₂ group, wherein R_{b} is independently H or alkyl, R_{c} is independently selected from the group consisting of alkoxy, amino, alkylamino and dialkylamino, R_{d} is selected from the group consisting of H, alkyl and —C(═O)Rₑ, wherein Rₑ is OH, alkoxy, amino, alkylamino, dialkylamino, hydrazino, alkylhydrazino, dialkylhydrazino or trialkylhydrazino;
and R₁ and R₂, independently, is H or β-D-galactopyranosyl group with the proviso that at least one of the R₁ and R₂ groups is H.

Even more preferably, X in donors of formulae 1, 2 or 3, independently, is selected from the group consisting of 4-nitrophenoxy, 2-nitrophenoxy, 2,4-dinitrophenoxy, 2-chloro-4-nitrophenoxy, lactose moiety, 2,5-dimethyl-3-oxo-(2H)-furan-4-yloxy, 2-ethyl-5-methyl-3-oxo-(2H)-furan-4-yloxy, 5-ethyl-2-methyl-3-oxo-(2H)-furan-4-yloxy, 4,6-dimethoxy-1,3,5-triazin-2-yloxy, 4,6-diethoxy-1,3,5-triazin-2-yloxy, or 4-methylumbelliferyloxy, or the donor of formula 4 is selected from compounds of formulae 5, 6 or 7.

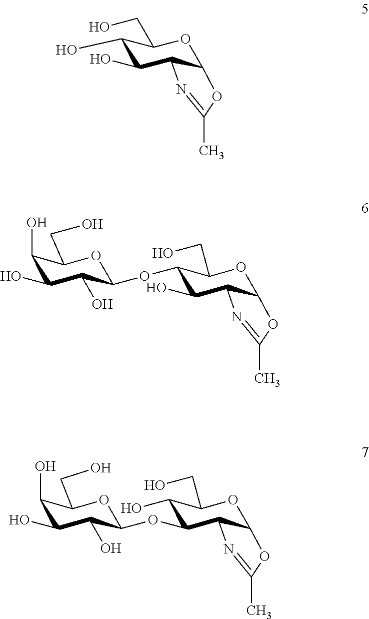

Particularly preferably, the donor of formula 1 is 3′-SL or 6′-SL. Likewise particularly preferably, the donor of formula 2 is 2′-FL.

In the present invention, if not otherwise indicated, different features of alternatives and embodiments can be combined with each other, where suitable.

Whilst the invention has been described with reference to a preferred embodiment, it will be appreciated that various modifications are possible within the scope of the invention.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used in the sense of ‘including’ rather than in to mean ‘consisting of’. All prior teachings acknowledged above are hereby incorporated by reference. No acknowledgement of any prior published document herein should be taken to be an admission or representation that the teaching thereof was common general knowledge in Australia or elsewhere at the date hereof.

### EXPERIMENTAL

### Example 1

### General Procedure for Transglycosylation Reactions

A solution of appropriate glycosyl donor(s) such as derivatives of general formula 1 to 4 and GOS (10 mM-1M) is incubated in an incubation buffer at a pH range from 5.0 to 9.0 with recombinant glycosidase, α-transglycosidase or α-glycosynthase, such as α-fucosidase, α-transfucosidase, α-fucosynthase, α-sialidase, α-transsialidase, β-N-acetylglucosaminidase, β-trans-N-acetylglucosaminidase, β-lacto-N-biosidase, β-trans-lacto-N-biosidase, β-N-acetyllactosaminidase or β-trans-N-acetyllactosaminidase. The reaction mixture is stirred at a temperature in the range of from 20 to 70° C. Samples are taken at different times of the reaction, the reaction is stopped by the addition of 1M NaHCO₃-solution at pH=10 and products are analysed by HPLC, or/and LC-MS, or/and LC-MS-MS. After completion, the enzyme is denatured and centrifuged. The resulting solution is evaporated under reduced pressure. After lyophilisation, the dry residue is dissolved in water and products are purified by biogel chromatography (P-2 Biogel, 16×900 mm) with water or by reverse phase chromatography.

The recombinant transglycosidases used in the examples 3 to 5 were produced in E. coli as reported by Osanjo et al. Biochemistry 46, 1022 (2007), Agusti et al. Glycobiology 14, 659 (2004), Neubacher et al. Org. Biomol. Chem. 3, 1551 (2005) and Sun et al. Biotechnol. Lett. 30, 671 (2008). The purified enzymes were stored between −20 and +4° C.

Commercially available GOS mixture was used in examples 3 to 5 (Vivinal GOS® [Friesland Foods Domo, The Netherlands] (Torres et al. Compr. Rev. Food Sci. Food Safety 9, 438 (2010), Hernández-Hernandez et al. J. Chromatogr. A 1220, 57 (2012)).

### Example 2

Manufacture of 2,5-dimethyl-3-oxo-(2H)-furan-4-yl α-L-fucopyranoside
a) The solution of 2,3,4-tri-O-(4-methoxybenzyl)-L-fucopyranose trichloroacetimidate (α/β mixture, prepared from 85 mmol of 2,3,4-tri-O-(4-methoxybenzyl)-L-fucopyranose and trichloroacetonitrile in the presence of NaH in quantitative yield) in diethyl ether (100 ml) was added to a mixture of 2,5-dimethyl-4-hydroxy-3-oxo-(2H)-furan (85 mmol) and TMS-triflate (1.2 ml) in diethyl ether (200 ml) at −14° C. After 3 hours the cooling bath was removed and the stirring continued for 1 hour. The reaction mixture was diluted with ethyl acetate and extracted with sat. NaHCO₃-solution (3×150 ml) and brine (150 ml). The organic phase was dried over Na₂SO₄ and evaporated. The resulting syrup was purified by column chromatography yielding 23.27 g of 2,5-dimethyl-3-oxo-(2H)-furan-4-yl 2,3,4-tri-O-(4-methoxybenzyl)-α-L-fucopyranoside as a thick yellow syrup in a 1:1 mixture of diastereoisomers. Selected NMR chemical shifts in CDCl₃: anomeric protons: 5.32 and 5.57 ppm, J_{1,2}=3.4 Hz; anomeric carbons: 113.81 and 113.95 Hz.
b) A suspension of the above product and Pd on charcoal (12.5 g) in ethyl acetate (1 l) was stirred at room temperature for 6 hours under H₂. The catalyst was filtered off and washed with ethyl acetate. The combined filtrate was evaporated to a solid mass which was suspended in ethyl acetate and allowed to stand at 5° C. for overnight. The crystalline material was filtered off and dried. The mother liquor was subjected to column chromatography. The combined products gave 5.50 g of 2,5-dimethyl-3-oxo-(2H)-furan-4-yl α-L-fucopyranoside as white solid in 1:1 mixture of diastereoisomers. Selected NMR chemical shifts in DMSO-d₆: anomeric protons: 5.07 and 5.52 ppm, J_{1,2}=2.6 Hz; anomeric carbons: 100.19 and 101.03 Hz. Purity by GC (after silylation): 98.9%.

### Example 3

### Fucosylation of GOS with 2′-FL as Donor

A solution of 2′-FL (20 mg) and GOS (75 mg) and was incubated in the presence of α-1,2-transfucosidase mutant P25 from Thermotoga maritima(Osanjo et al. Biochemistry 46, 1022 (2007), 94 μg) according to the general procedure in an incubation buffer (50 mM citrate-phosphate, 145 mM NaCl, pH 5.5, 190 μl) at 60° C. for 24 hours.

Qualitative LC-MS analysis was carried out to identify new fucosylated galactooligosaccharides based on the method described by Hernández-Hernandez et al. J. Chromatogr. A 1220, 57 (2012).

### Experimental Conditions

Instrument: Bruker microQTof II MS coupled with Dionex Ultimate 3000 UHPLC
Ionisation: ESI negative
Dry temperature: 200° C.
Calibration: with Na-formate cluster solution

### Applied HPLC Method:

| Column | XBridge ™ Amide 3.5um, 4.6 × 250 mm | | |
|---|---|---|---|
| Temperature oven (° C.) | 35 | | |
| Flow (ml/min) | 1.0, split to MS 1:1 | | |
| Mobile phase | A: water with 0.1% ammonium hydroxide | | |
| | B: acetonitrile with 0.1% ammonium hydroxide | | |
| Elution type: | gradient | | |
| | time [min] | A [%] | B [%] |
| Gradient profile: | 0 | 20 | 80 |
| | 35 | 50 | 50 |
| | 40 | 50 | 50 |
| | 40.5 | 20 | 80 |
| | 45 | 20 | 80 |

| | |
|---|---|
| Test time (min): | 45 |
| Autosampler temperature | 10 |
| (° C.): | |
| Injected volume (μl) | 5 |

### Fucosylated Oligosaccharides Detected:

| oligosaccharides | retention time (min) |
|---|---|
| Fuc-[(Gal)₂-A] (A: Glc or Gal); [M − H]⁻ = 649 | 19.33, 19.90, 21.09, |
| | 21.56, 21.94, 22.21, |
| | 22.73, 23.25 |
| Fuc-[(Gal)₃-A] (A: Glc or Gal); [M − H]⁻ = 811 | 22.58, 23.28, 23.60, |
| | 24.21, 24.64, 25.04, |
| | 25.35, 25.58, 26.20 |
| Fuc-[(Gal)₄-A] (A: Glc or Gal); [M − H]⁻ = 973 | 27.57, 28.40 |
| Fuc-[(Gal)₅-A] (A: Glc or Gal); [M − H]⁻ = 1135 | 30.44 |
| (Fuc)₂-[(Gal)₂-A] (A: Glc or Gal); [M − H]⁻ = 796 | 21.00, 22.04, 22.31, |
| | 22.76, 23.30, 23.97, |
| | 24.36 |

### Example 4

### Sialylation of GOS with 3′-SL as Donor

A solution of 3′-SL (12.7 mg) and GOS (75 mg) and was incubated in the presence of α-2,3-transsialidase from T. cruzi (GenBank Database gi|432485, 4 μg) according to the general procedure in an incubation buffer (100 mM tri-HCl, pH 7.5, 180 μl) at 37° C. for 24 hours. Qualitative LC-MS analysis was carried out to identify new sialylated galactooligosaccharides (see Example 3 for conditions).

### Sialylated Oligosaccharides Detected:

| | retention |
|---|---|
| oligosaccharides | time (min) |
| Neu5Ac-[(Gal)₂-A] (A: Glc or Gal); [M − H]⁻ = 794 | 4.13, 4.56, |
| | 5.13, 5.43 |
| Neu5Ac-[(Gal)₃-A] (A: Glc or Gal); [M − H]⁻ = 956 | 5.65, 6.17, |
| | 6.62, 7.06, |
| | 7.41 |
| Neu5Ac-[(Gal)₄-A] (A: Glc or Gal); [M − H]⁻ = 1118 | 7.29, 7.71, |
| | 8.27, 8.53, |
| | 8.75, 9.00 |
| Neu5Ac-[(Gal)₅-A] (A: Glc or Gal); [M − H]⁻ = 1280 | 9.12, 9.67, |
| | 10.34 |
| (Neu5Ac)₂-[(Gal)₂-A] (A: Glc or Gal); [M − 2H]²⁻ = 542 | 2.58 |
| (Neu5Ac)₂-[(Gal)₃-A] (A: Glc or Gal); [M − 2H]²⁻ = 623 | 2.63 |
| (Neu5Ac)₂-[(Gal)₄-A] (A: Glc or Gal); [M − 2H]²⁻ = 704 | 3.42 |
| (Neu5Ac)₂-[(Gal)₅-A] (A: Glc or Gal); [M − 2H]²⁻ = 785 | 4.70 |

Due to the regioselectivity of the enzyme used, the products are assumed to have α2-3-sialyl residues.

### Example 5

### Sialylation of GOS with 6′-SL as Donor

A solution of 6′-SL (12.7 mg) and GOS (75 mg) and was incubated in the presence of recombinant truncated α-2,6-transsialidase Δ15Pd2,6ST from Photobacterium damsela (Cheng et al. Glycobiology 20, 260 (2010), 60 μg) according to the general procedure in an incubation buffer (100 mM phosphate buffer, pH 6.0, 190 μl) at 37° C. for 24 hours.

Qualitative LC-MS analysis was carried out to identify new sialylated galactooligosaccharides (see Example 3 for conditions).

### Sialylated Oligosaccharides Detected:

| oligosaccharides | retention time (min) |
|---|---|
| Neu5Ac-[(Gal)₂-A] (A: Glc or Gal); | 8.30, 8.67, 8.85, 9.24, 9.62, |
| [M − H]⁻ = 794 | 10.03, 10.53, 11.62 |
| Neu5Ac-[(Gal)₃-A] (A: Glc or Gal); | 10.73, 11.05, 11.62, 12.04, |
| [M − H]⁻ = 956 | 12.41, 12.84 |
| Neu5Ac-[(Gal)₄-A] (A: Glc or Gal); | 12.87, 13.18, 13.70, 13.91, |
| [M − H]⁻ = 1118 | 14.25, 14.48, 14.62, |
| | 15.02, 15.47 |
| Neu5Ac-[(Gal)₅-A] (A: Glc or Gal); | 15.44, 16.06, 16.24, 16.44, |
| [M − H]⁻ = 1280 | 16.70 |
| (Neu5Ac)₂-[(Gal)₂-A] (A: Glc or Gal); | 3.34 |
| [M − 2H]²⁻ = 542 | |
| (Neu5Ac)₂-[(Gal)₃-A] (A: Glc or Gal); | 7.34, 7.91, 8.23, 8.77 |
| [M − 2H]²⁻ = 623 | |
| (Neu5Ac)₂-[(Gal)₄-A] (A: Glc or Gal); | 8.95, 9.39, 9.69, 9.87, 10.18 |
| [M − 2H]²⁻ = 704 | |
| (Neu5Ac)₂-[(Gal)₅-A] (A: Glc or Gal); | 9.81, 10.33, 10.66, 11.25 |
| [M − 2H]²⁻ = 785 | |

Due to the regioselectivity of the enzyme used, the products are assumed to have α2-6-sialyl residues.

1. A method for making a modified galactooligosaccharide or a mixture of modified galactooligosaccharides, characterized in that at least one glycosyl donor, which is not a galactosyl donor, is reacted with a precursor galactooligosaccharide represented by the formula (Gal)ₙ-A, wherein A means galactose or glucose, and n is at least 2, or a mixture of precursor galactooligosaccharides, under the catalysis of an enzyme capable of transferring said glycosyl moiety to said precursor galactooligosaccharide or mixture of precursor galactooligosaccharides.

2. The method according to claim 1, comprising the steps of
a) providing at least one glycosyl donor,
b) providing a precursor galactooligosaccharide or a mixture of precursor galactooligosaccharides,
c) providing at least one enzyme comprising a trans-glycosidase or a glycosynthase activity;
d) preparing a mixture of the components provided in steps a), b) and c);
e) incubating the mixture prepared according to step d);
f) optionally: repeating steps a), c), d) and e) with the mixture obtained according to step e).

3. (canceled)

4. The method according to claim 1, wherein the at least one glycosyl donor is selected from the group consisting of a sialyl donor, a fucosyl donor and an optionally galactosylated N-acetyl-glucosaminyl donor, and the enzyme comprises a fucosidase, trans-fucosidase or fucosynthase activity, a sialidase (neuraminidase) or trans-sialidase (transneuraminidase) activity, a N-acetylglucosaminidase or trans-N-acetylglucosaminidase activity, a lacto-N-biosidase or trans-lacto-N-biosidase activity and/or a N-acetyllactosaminidase or trans-N-acetyllactosaminidase activity.

5. The method according to claim 4, wherein the donor is 3′-SL and the enzyme comprises α2-3-trans-sialidase activity.

6. The method according to claim 1, wherein the at least one glycosyl donor is selected from the group consisting of a sialyl donor, a fucosyl donor and an optionally galactosylated N-acetyl-glucosaminyl donor, and the enzyme comprises a fucosidase, trans-fucosidase or fucosynthase activity, a sialidase (neuraminidase) or trans-sialidase (transneuraminidase) activity, a N-acetylglucosaminidase or trans-N-acetylglucosaminidase activity, a lacto-N-biosidase or trans-lacto-N-biosidase activity and/or a N-acetyllactosaminidase or trans-N-acetyllactosaminidase activity, provided that the sialidase or trans-sialidase activity is not a α2-3-sialidase or α2-3-trans-sialidase activity.

7. (canceled)

8. The method according to claim 6, wherein the sialyl donor is characterized by formula 1, and/or the fucosyl donor is characterized by formula 2, and/or the optionally galactosylated N-acetyl-glucosaminyl donor is characterized by formulae 3 or 4 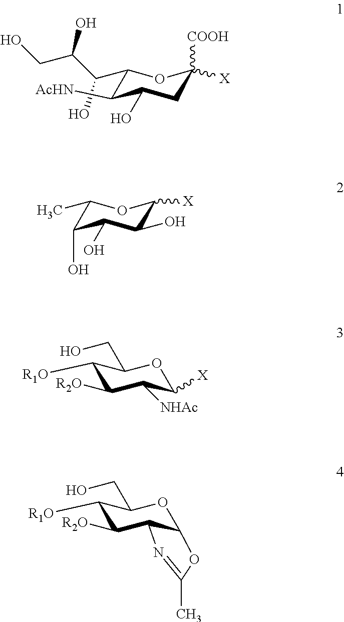
wherein X, independently, is selected from the group consisting of 4-nitrophenoxy, 2-nitrophenoxy, 2,4-dinitrophenoxy, 2-chloro-4-nitrophenoxy, lactose moiety, 2,5-dimethyl-3-oxo-(2H)-furan-4-yloxy, 2-ethyl-5-methyl-3-oxo-(2H)-furan-4-yloxy, 5-ethyl-2-methyl-3-oxo-(2H)-furan-4-yloxy, 4,6-dimethoxy-1,3,5-triazin-2-yloxy, 4,6-diethoxy-1,3,5-triazin-2-yloxy, or 4-methylumbelliferyloxy;
and R₁ and R₂, independently, is H or β-D-galactopyranosyl group with the proviso that at least one of the R₁ and R₂ groups is H.

9. (canceled)

10. The method according to claim 8, wherein the donor of formula 1 is 6′-SL.

11. The method according to claim 6, wherein the enzyme comprises α-trans-fucosidase activity and wherein the glycosyl donor is fucosyl.

12. The method according to claim 11, wherein the glycosyl donor is 2′-FL.

13. The method according to claim 1, for enhancing the bifidogenic effect of galactooligosaccharides.

14. A modified galactooligosaccharide or a mixture of modified galactooligosaccharides obtainable according to claim 6.

15. A modified galactooligosaccharide according to claim 14 comprising at least one glycosyl residue selected from the group consisting of sialyl (expect except for α2-3-sialyl), fucosyl, N-acetylglucosaminyl, N-acetyllactosaminyl, lacto-N-biosyl, sialylated and/or fucosylated N-acetylglucosaminyl, N-acetyllactosaminyl and lacto-N-biosyl moieties further glycosylated with sialyl and/or fucosyl and/or N-acetyllactosaminyl and/or lacto-N-biosyl and/or N-acetylglucosaminyl, wherein a precursor galactooligosaecharide represented by the formula (Gal)ₙ-A, in which formula A is galactose or glucose, and n is at least 2, is coupled to at least one glycosyl residue via the anomeric carbon atom of the glycosyl residue, to any of the monosaccharide units of said precursor galactooligosaccharide.

16-17. (canceled)

18. The modified galactooligosaecharide according to claim 11, wherein the at least one glycosyl residue is fucosyl linked with 1-2, 1-3, 1-4 or 1-6 interglycosidic linkage.

19. (canceled)

20. A modified galactooligosaccharide comprising at least one glycosyl residue, wherein a precursor galactooligosaccharide represented by the formula (Gal)ₙ-A, in which formula A means galactose or glucose and n is at least 2, is coupled to at least one glycosyl residue via the anomeric carbon atom of the glycosyl residue, to any of the monosaccharide units of said precursor galactooligosaccharide, wherein said glycosyl residue is not galactosyl.

21. The compound according to claim 20, wherein the at least one glycosyl moiety is selected from the group consisting of sialyl, fucosyl and glycosylated or unglycosylated N-acetyl-glucosaminyl moiety, and n ranges from 2 to 15.

22. The compound according to claim 20, wherein the at least one glycosyl residue is sialyl linked with 2-3 or 2-6 interglycosidic linkage.

23-24. (canceled)

25. The compound according to claim 20, wherein the at least one glycosyl residue is fucosyl linked with 1-2, 1-3, 1-4 or 1-6 interglycosidic linkage.

26. (canceled)

27. The compound according to claim 20, wherein the at least one glycosyl residue is glycosylated or unglycosylated N-acetyl-glucosaminyl linked with 1-3, 1-4 or 1-6 interglycosidic linkage.

28-30. (canceled)

31. A mixture of at least two compounds according to claim 20.

32-34. (canceled)

35. A consumable product comprising a modified galactooligosaccharide according to claim 20 or a mixture of at least two of said modified galactooligosaccharides, and nutritionally and/or pharmaceutically acceptable carriers.

36. (canceled)

37. The consumable product according to claim 35, which is an infant, follow-up or toddler formula.
